SEQUENCE LISTING (36 sequences)

SEQ ID NO: 1 - DNA, 1239 bp, Plasmodium falciparum
  CDS (1)..(1239)
atgaatttattaaaattaattggaaaaaataagaaaatgaagaatgaaaatatgggtaattccatattttattattcctgttatgttattatagttcttactataatattgtctaagtttgttgtaatccctttgatggctcaaatgtttttgtacacattcattacaatatatattggaagtcatgatagtttgaaacaattagaaattgatgataaaaccaaaaagtcagacaacataacagcctatgatgctatgatgtttccagtaattggatctgcagctttgcttactttatatttcgcatataagttcttagatccgttttatgtgaatttattattgactctttacctaaccttggcgggtgtattttccttacagggtgtatttacaacaatcttggaacctgtttttccaaatttttttaaaaaagatgaatatgtcaaaacattcaaattaccaaattttatatataaagaacctattgtattcaatactaataaaggagaaatagtttgcttaatactcagctttgctataggattgcgttggatattttataaagacttcattacacataacgttttggcagtttctttttgttttcaagccatatctttggtaattcttagcaactttttaataggattcttattattatctggtttgtttgtatatgatattttctgggtttttggaaacgatgttatggttacagtagctaagtcttttgaagctccagtaaaattgttattcccagtttcgagtgatccagtacattacagtatgcttggtttaggagatattattataccaggaatattgatgtctttatgtttacgttttgattattatttatttaagaataacatacataaaggaaacttaaagaaaatgtttaatgatatatctatacatgaatctttcaagaaatattatttttataccattataatattttacgaattaggtttagttgttacatattgtatgctcttttattttgaacatcctcaaccagctcttctttatttggtacctgcatgtatacttgccatattagcttgttccatatgcaaaagagaatttaaattaatgataaaatatcaagaaattacagacaaatccaatactgtagatgatgcaagtaagaataaaaaaaaagataaggaagaaatccccaaaattcaagagaccccagtgtcaaatgcaaaaaaaagaattaccaataaatga

SEQ ID NO: 2 - protein, 412 aa, Plasmodium falciparum
MNLLKLIGKNKKMKNENMGNSIFYYSCYVIIVLTIILSKFVVIPLMAQMFLYTFITIYIGSHDSLKQLEIDDKTKKSDNITAYDAMMFPVIGSAALLTLYFAYKFLDPFYVNLLLTLYLTLAGVFSLQGVFTTILEPVFPNFFKKDEYVKTFKLPNFIYKEPIVFNTNKGEIVCLILSFAIGLRWIFYKDFITHNVLAVSFCFQAISLVILSNFLIGFLLLSGLFVYDIFWVFGNDVMVTVAKSFEAPVKLLFPVSSDPVHYSMLGLGDIIIPGILMSLCLRFDYYLFKNNIHKGNLKKMFNDISIHESFKKYYFYTIIIFYELGLVVTYCMLFYFEHPQPALLYLVPACILAILACSICKREFKLMIKYQEITDKSNTVDDASKNKKKDKEEIPKIQETPVSNAKKRITNK

SEQ ID NO: 3 - protein, 41 aa, Artificial Sequence
  Synthetic
VYDIFWVFGNDVMVTVAKSFEAPVKLLFPVSSDPVHYSMLG

SEQ ID NO: 4 - protein, 19 aa, Artificial Sequence
  Synthetic
EAPVKLLFPVSSDPVHYSM

SEQ ID NO: 5 - protein, 20 aa, Artificial Sequence
  Synthetic
EIPKIQETPVSNAKKRITNK

SEQ ID NO: 6 - protein, 230 aa, Artificial Sequence
  Synthetic
LRWIFYKDFITHNVLAVSFCFQAISLVILSNFLIGFLLLSGLFVYDIFWVFGNDVMVTVAKSFEAPVKLLFPVSSDPVHYSMLGLGDIIIPGILMSLCLRFDYYLFKNNIHKGNLKKMFNDISIHESFKKYYFYTIIIFYELGLVVTYCMLFYFEHPQPALLYLVPACILAILACSICKREFKLMIKYQEITDKSNTVDDASKNKKKDKEEIPKIQETPVSNAKKRITNK

SEQ ID NO: 7 - protein, 42 aa, Artificial Sequence
  Synthetic
GMPWLSATTVRSVTHANALTVMGKASTPGAAAQIQEVKEQRI

SEQ ID NO: 8 - DNA, 30 bp, Artificial Sequence
  Synthetic
gccggatccatgaatttattaaaattaatt

SEQ ID NO: 9 - DNA, 29 bp, Artificial Sequence
  Synthetic
gccgtcgactcatttattggtaattcttt

SEQ ID NO: 10 - protein, 413 aa, Plasmodium vivax
MNLLKLISKNNKKMKNDNMGCSIVYYSSYVIILLTIVLSKFFVIPLMAQMFLYTFITIYIGSHDSLKQLEQVDDRNKKADNITAYDAIMFPIIGSGALLTLYFAYKFLDPYYVNMLLTVYLTLAGVFSLQGVCANILEPALPKFFKKDEYVKTFKLPGFISKEPVIFNTNKGEIISFLFCFFIGARWIFYKDFITHNVLAVSFCFQAISLVILSNFLIGFLLLSGLFVYDIFWVFGNDVMVTVAKSFEAPVKLLFPVSKDPVHYSMLGLGDIIIPGIVISLCLRFDYYLHRNKIHKGNFKKMFNDISIHEAFKKYYFFTISVFYQIGLVVTYCMLFYFEHAQPALLYLVPACILAIVGCSLCKGEFKIMVKYQEITDKSNSSDDGKKKVADKDEMYKSQESIISNAKKRITNK

SEQ ID NO: 11 - protein, 413 aa, Plasmodium knowlesi
MNLLKLISKNNKKMKSDSMGYSIFYYSSYVIILLTIVLSKFFVIPLMVQMFLYTFITIYIGSHDSLKQLEQVEDKNKKADNITAYDAIMFPIIGSGALLTLYFAYKFLDPYYVNMLLTVYLTLAGIFSLQGACANILEPAFPKFFKKDEYVKTFKLPGFISKEPVVFNTNKGEIISFLVCFFIGGRWIFYKDFITHNVLAVSFCFQAISLVILSNFLIGFLLLSGLFVYDIFWVFGNDVMVTVAKSFEAPVKLLFPVSTDPVHYSMLGLGDIIIPGIVISLCLRFDYYLHRNKIHKGNFKKMFNDISIHESFKKYYFYTITVFYQLGLVVTYCMLFYFEHAQPALLYLVPACILAIVGCSLFKGEFKMMVKYQEITDKSSASDDGKKKVADKDEMFKSQESIISNAKKRITNK

SEQ ID NO: 12 - protein, 405 aa, Plasmodium berghei
MNLLKLIGKNKKMKNHDKRNSAIYYSCYAIIVLTIILSRFIVIPLILQMTLYTFITIYIGSHESIRQLEADDKTQKTDHITTYDAIMFPIIGSAALLTLYFAYKFLDPYYVNLLLTVYLTMAGVFSLQSEPFFPKIFKKDEFVKTINAPKFISKDPIVFNTNKGEIMSLIVCFIIGARWIFYKDFVTHNILAISFCFQALSLVILSNFVIGFILLSGLFVYDIFWVFGNDVMVTVAKSFEAPVKLLFPVSLDPLHYSMLGLGDIIIPGILISLCLRFDYYLHRNKIHKGNVKKMFNDISIHESFKKYYFYTITVFYQAGLILTYCMLFYFEHAQPALLYLVPACILAIVGCALFKKEFKIMIKYQEITDKSSNADDGKKKTLEKEETLKSQESIMSVTKRKINAK

SEQ ID NO: 13 - DNA, 27 bp, Artificial Sequence
  Synthetic
cgcgaattcgtatatgatattttctgg

SEQ ID NO: 14 - DNA, 27 bp, Artificial Sequence
  Synthetic
cgctctagaaccaagcatactgtaatg

SEQ ID NO: 15 - DNA, 24 bp, Artificial Sequence
  Synthetic
acagtctggtttgtttgtatatga

SEQ ID NO: 16 - DNA, 29 bp, Artificial Sequence
  Synthetic
ctggtataataatatctcctaaaccaagc

SEQ ID NO: 17 - DNA, 22 bp, Artificial Sequence
  Synthetic
atacatattaattgttcttgtt

SEQ ID NO: 18 - DNA, 20 bp, Artificial Sequence
  Synthetic
ttgaagctccagtaaaattg

SEQ ID NO: 19 - DNA, 31 bp, Artificial Sequence
  Synthetic
ggcttccgcggatgaatttattaaaattaat

SEQ ID NO: 20 - DNA, 31 bp, Artificial Sequence
  Synthetic
tacagcttaagagtaagcaaagctgcagatc

SEQ ID NO: 21 - DNA, 28 bp, Artificial Sequence
  Synthetic
gccgaattctctggtttgtttgtatatg

SEQ ID NO: 22 - DNA, 29 bp, Artificial Sequence
  Synthetic
gccgaattctcatttattggtaattcttt

SEQ ID NO: 23 - protein, 20 aa, Artificial Sequence
  Synthetic
GNSIFYYSCYVIIVLTIILS

SEQ ID NO: 24 - protein, 5 aa, Artificial Sequence
  Synthetic
LGLGD

SEQ ID NO: 25 - protein, 17 aa, Artificial Sequence
  Synthetic
SIFYYSCYVIIVLTIIL

SEQ ID NO: 26 - protein, 17 aa, Artificial Sequence
  Synthetic
KFVVIPLMAQMFLYTFI

SEQ ID NO: 27 - protein, 21 aa, Artificial Sequence
  Synthetic
YDAMMFPVIGSAALLTLYFAY

SEQ ID NO: 28 - protein, 21 aa, Artificial Sequence
  Synthetic
LLLTLYLTLAGVFSLQGVFTT

SEQ ID NO: 29 - protein, 21 aa, Artificial Sequence
  Synthetic
KGEIVCLILSFAIGLRWIFYK

SEQ ID NO: 30 - protein, 21 aa, Artificial Sequence
  Synthetic
VLAVSFCFQAISLVILSNFLI

SEQ ID NO: 31 - protein, 21 aa, Artificial Sequence
  Synthetic
LLSGLFVYDIFWVFGNDVMVT

SEQ ID NO: 32 - protein, 21 aa, Artificial Sequence
  Synthetic
SMLGLGDIIIPGILMSLCLRF

SEQ ID NO: 33 - protein, 21 aa, Artificial Sequence
  Synthetic
YFYTIIIFYELGLVVTYCMLF

SEQ ID NO: 34 - protein, 21 aa, Artificial Sequence
  Synthetic
PALLYLVPACILAILACSICK

SEQ ID NO: 35 - protein, 40 aa, Artificial Sequence
  Synthetic
EQKLISEEDLMDSKGSSQKGSRLLLLLVVSNLLLCQGVVS

SEQ ID NO: 36 - protein, 40 aa, Artificial Sequence
  Synthetic
EQKLISEEDLMDSKGSSQKGSRLLLLLVVSNLLLCQGPPS

(19) United States
(12) Patent Application Publication
(10) Pub. No.: US 2011/0171225 A1
(43) Pub. Date: Jul. 14, 2011

(54) Novel Plasmodium Falciparum Gene Encoding Signal Peptide Peptidase and Method of Using Inhibitors Thereof for Inhibiting Malarial Infection

(75) Inventors: Athar H. Chishti, Darien, IL (US); Xuerong Li, Chicago, IL (US)

(73) Assignee: The Board of Trustees of the University of Illinois, Urbana, IL (US)

(21) Appl. No.: 13/063,723

(22) PCT Filed: Sep. 14, 2009
(86) PCT No.: PCT/US09/56836

§ 371 Date: Mar. 11, 2011

Related U.S. Application Data
(60) Provisional application No. 61/096,592, filed on Sep. 12, 2008.

(51) Int. Cl. A61K 39/395; A61K 39/002; A61K 38/48; G01N 33/573; C12Q 1/37; C12N 9/48; C12N 5/10; C12N 5/02; C12N 1/21; C12N 1/19; C12N 1/10; C12N 15/63; A61K 38/00; A61P 33/06; C07K 16/40; C07H 21/04; A61P 33/02

(52) U.S. Cl. 424/139.1; 424/191.1; 424/94.63; 435/7.4; 435/24; 435/212; 435/325; 435/348; 435/375; 435/252.3; 435/254.2; 435/258.2; 435/320.1; 514/4.4; 530/387.3; 530/387.9; 536/23.2

(57) ABSTRACT

This invention provides reagents, methods and pharmaceutical compositions for treating and preventing malaria. Specifically, the invention provides methods for inhibiting a Plasmodium parasite, especially Plasmodium falciparum, from invading or replicating in a cell as well as vaccines for preventing malaria.

This invention relates to and claims the benefit of priority to U.S. Provisional Application Ser. No. 61/096,592 filed on Sep. 12, 2008, the disclosure of which is herein incorporated by reference in its entirety.

This invention is supported in part by Grant Nos. HL 60961 and AL054532 from the National Institute of Health (NIH). Thus, the United States Government has certain rights in the invention.

### BACKGROUND OF THE INVENTION

1. Field of the Invention

This application relates to the protozoan parasite Plasmodium, especially Plasmodium falciparum and the signal peptide peptidase of the parasite. Specifically, the application relates to compositions, methods, and reagents useful for inhibiting Plasmodium infection or replication as a malaria treatment in patients based on the Plasmodium falciparum signal peptide peptidase (PfSPP).

2. Description of Related Art

Malaria is one of the most common infectious diseases globally with significant morbidity, mortality and economic consequences. It is caused by a protozoan parasite of the genus Plasmodium that infects 300-500 million people and causes an estimated 2 million deaths annually (Andrews et al., 2006, Antimicro. Agents Chemotherapy 50:638-48). Despite decades of research efforts, malaria, especially malaria caused by Plasmodium falciparum having the highest rates of complications and mortality, continues to be one of the most widespread and prevalent diseases today.

Plasmodium falciparum is transmitted to humans by the bite of females of the Anopheles mosquitoes. The infection is initiated when sporozoites are inoculated into the patient's blood stream from the saliva of an infected mosquito vector. The sporozoites invade parenchymal cells of the liver and develop into merozoites. One to two weeks after the initial infection, the hepatocytes burst and merozoites are released. The released merozoites invade red blood cells (RBCs) by a process involving multiple ligand-receptor interactions between the parasite proteins and the surface proteins on the RBCs.

The parasite first binds to the erythrocytes in a random orientation. It then reorients such that the apical complex of the merozoite is in proximity to the erythrocyte membrane. During the process, two apical organelles, micronemes and rhoptries, rapidly secrete their contents when the merozoite apical end comes in close proximity to the RBC membrane (Li et al., 2008, Mol. Biochem. Parasitology 158:22-31). It is believed that proteolysis of several RBC proteins and Plasmodium proteins occurs during host cell invasion, and several Plasmodium intramembrane serine proteases have been implicated in these processes (O'Donnell et al., 2005, Curr. Opin. Micro. 8:422-427). Subsequently, a tight junction is formed between the parasite and erythrocyte. As it enters the erythrocyte, the parasite forms a parasitophorous vacuole in which the parasite continues to develop during the blood stage of its life cycle.

The clinical manifestations of malaria are directly linked to the blood-stage lifecycle of Plasmodium parasites, in which the parasites proliferate asexually within the host RBCs. In the RBCs, merozoites develop sequentially into ring forms, trophozoites, and schizonts, each of which expresses both shared and unique antigens. The blood stage of the life cycle continues when schizont-infected RBCs burst and release merozoites that invade other erythrocytes. Sexual stage gametocytes develop in some erythrocytes and are taken up by mosquitoes during a blood meal, after which they fertilize and develop into oocysts. Within two weeks in the mosquito vector, immature sporozoites derived from the oocysts develop and travel to the salivary glands, where they mature and become infectious.

The clinical features of malaria include fever spikes, shivering, anemia, vomiting, retinal damage, hemoglobinuria, and splenomegaly. Infected erythrocytes are often sequestered in various human tissues or organs due to the interactions of host cell receptors and parasite-derived proteins present on the RBC membrane. Sequestration of infected erythrocytes in the brain causes the often fatal cerebral malaria, to which children are most vulnerable.

Potential approaches to control malaria include vaccine development, vector control, and drug treatment (Andrews et al. 2006, supra). Effective vaccine development has been hampered by immune evasion as a result of parasite antigen variation. Although insecticide spraying has reduced the incidence of the disease and parasite transmissions in certain regions of the world, rising insecticide resistance has limited the effectiveness of this approach. Chloroquine has been the most widely used anti-malaria drug; however, emerging drug resistance highlights the urgent need of identifying new drug targets against malaria. Thus, there exists a need in the anti-malarial arts for a better treatment and a better drug target for treating malaria.

### SUMMARY OF THE INVENTION

This invention provides reagents, methods and pharmaceutical compositions for treating and preventing malaria in humans. Specifically, the invention provides reagents and methods for inhibiting Plasmodium invasion and replication in cells, especially red blood cells, and vaccines for preventing malaria. Plasmodium species relating to the reagents and methods of the invention include Plasmodium falciparum, Plasmodium vivax, Plasmodium malariae, and Plasmodium ovale.

In one aspect, the invention provides isolated nucleic acids comprising a polynucleotide sequence: (a) that is identified by SEQ ID NO:1; (b) that encodes a polypeptide comprising the amino acid sequence identified by SEQ ID NO:2, or (c) that is complementary to the nucleotide sequence of (a) or (b). In certain embodiments, the isolated nucleic acids comprise a polynucleotide sequence that encodes a polypeptide having the amino acid sequence as identified by SEQ ID NO:2.

In other aspects, the invention provides purified preparations of a polypeptide having an amino acid sequence identified by SEQ ID NO:2 having Plasmodium falciparum signal peptide peptidase activity. In certain particular embodiments, the preparations comprise lipids, including phospholipids, to increase the solubility and/or activity of the PfSPP protein.

In another aspect, the invention provides membrane preparations comprising a polypeptide having an amino acid sequence identified by SEQ ID NO:2 having Plasmodium falciparum signal peptide peptidase activity.

In a further aspect, the invention provides expression vectors comprising nucleic acids encoding a Plasmodium falciparum signal peptide peptidase as disclosed herein.

In another aspect, the invention further provides a host cell comprising an expression vector of the invention encoding a Plasmodium falciparum signal peptide peptidase as disclosed herein. In certain embodiments, the host cell is a bacteria cell, a mammalian cell, a yeast cell, or an insect cell.

In yet another aspect, the invention provides methods for expressing a Plasmodium falciparum signal peptide peptidase as disclosed herein comprising the steps of culturing a host cell of the invention under conditions suitable for expressing the Plasmodium falciparum signal peptide peptidase as disclosed herein. In certain embodiments of this aspect, the polypeptide comprises an amino acid sequence identified by SEQ ID NO:2.

In a further aspect, the invention provides purified antibodies or antigen-binding fragments thereof that specifically bind to a Plasmodium falciparum signal peptide peptidase comprising the amino acid sequence identified by SEQ ID NO:2. In particular embodiments, the antibodies or antigen-binding fragments thereof recognize an epitope located within amino acid residues 246-264 of SEQ ID NO:2 (SEQ ID NO:4). In alternative particular embodiments, the antibodies or antigen-binding fragments thereof recognize an epitope located within amino acid residues 393-412 of SEQ ID NO: 2 (SEQ ID NO:5). In yet other embodiments, the antibodies of the invention are polyclonal antibodies or antigen-binding fragments thereof or in particular embodiments are monoclonal antibodies or antigen-binding fragments thereof. In yet other embodiments of the aspect, the antibodies are humanized, human, chimeric, or CDR-grafted antibodies or antigen-binding fragments thereof. In still other embodiments, the antibodies or antigen-binding fragments thereof of the invention inhibit the binding to an erythrocyte of a Plasmodium falciparum signal peptide peptidase as disclosed herein. In particular embodiments, the antibodies or antigen-binding fragments thereof inhibit the binding of a Plasmodium falciparum signal peptide peptidase as disclosed herein to the erythrocyte surface protein band 3.

In a further aspect, the invention provides methods of inhibiting a Plasmodium parasite invasion of a cell, comprising contacting the Plasmodium parasite with an antibody of the invention immunologically specific for a Plasmodium falciparum signal peptide peptidase as disclosed herein. In certain particular embodiments, the cell is an erythrocyte. In other embodiments, the antibody or antigen-binding fragment thereof recognizes an epitope located within amino acid residues 246-264 of SEQ ID NO: 2. In certain particular embodiments, the Plasmodium parasite is Plasmodium falciparum.

In yet another aspect, the invention provides methods of inhibiting a Plasmodium parasite replication, growth or development in a cell comprising contacting the cell with an antibody as described herein immunologically specific for a Plasmodium falciparum signal peptide peptidase as disclosed herein. In certain embodiments, the antibodies enter the Plasmodium-infected RBCs by mild detergent treatment of the cells. In certain embodiments, the methods further comprise contacting the cell with an effective amount of signal peptide peptidase (SPP) inhibitor. In particular embodiments, the signal peptide peptidase inhibitor is (Z-LL)₂-ketone, LY411575, NVP-AHW700-NX, or L685,458. In certain particular embodiments, the Plasmodium is Plasmodium falciparum.

In still another aspect, the invention provides methods of treating or preventing malaria in a human in need thereof comprising administering to the human an effective amount of a purified antibody or antigen-binding fragment thereof as described herein immunologically specific for a Plasmodium falciparum signal peptide peptidase as disclosed herein. In certain embodiments, the methods further comprise administering to the human an effective amount of an inhibitor of a Plasmodium falciparum signal peptide peptidase as disclosed herein. In particular embodiments, the SPP inhibitor is (Z-LL)₂-ketone, LY411575, NVP-AHW700-NX or L685,458.

In another aspect, the invention provides methods of inhibiting a Plasmodium parasite invasion of a cell comprising contacting the Plasmodium parasite, particularly a Plasmodium falciparum parasite, with an inhibitor of a Plasmodium falciparum signal peptide peptidase as disclosed herein. In certain embodiments, the inhibitor is (Z-LL)₂-ketone, LY411575, NVP-AHW700-NX or L685,458. In other embodiments, the methods further comprise contacting the Plasmodium parasite with an antibody or antigen-binding fragment thereof as described herein immunologically specific for a Plasmodium falciparum signal peptide peptidase as disclosed herein. In certain particular embodiments, the Plasmodium is Plasmodium falciparum.

In a further aspect, the invention provides methods of inhibiting a Plasmodium parasite replication, growth or development in a cell comprising contacting the cell with an inhibitor of a Plasmodium falciparum signal peptide peptidase as disclosed herein. In certain embodiments, the inhibitor is (Z-LL)₂-ketone, LY411575, NVP-AHW700-NX or L685,458. In other embodiments, the methods further comprise contacting the cell with an antibody or antigen-binding fragment thereof as described herein immunologically specific for a Plasmodium falciparum signal peptide peptidase as disclosed herein. In certain particular embodiments, the Plasmodium is Plasmodium falciparum.

In yet another aspect, the invention provides methods of treating or preventing malaria in a human in need thereof comprising administering to the human an effective amount of an inhibitor of a Plasmodium falciparum signal peptide peptidase as disclosed herein. In certain embodiments, the methods further comprise administering to the human an effective amount of an antibody or antigen-binding fragment thereof as described herein immunologically specific for a Plasmodium falciparum signal peptide peptidase as disclosed herein. In particular embodiments, the inhibitor is (Z-LL)2-ketone, LY411575, NVP-AHW700-NX or L685,458.

In a further aspect, the invention provides pharmaceutical compositions for inhibiting or preventing malaria comprising an antibody of the invention or antigen-binding fragment thereof immunologically specific for a Plasmodium falciparum signal peptide peptidase as disclosed herein and at least one pharmaceutically acceptable carrier, diluent, and excipient. In certain embodiments, the pharmaceutical compositions further comprise an inhibitor of a Plasmodium falciparum signal peptide peptidase as disclosed herein. In particular embodiments, the inhibitor is (Z-LL)2-ketone, LY411575, NVP-AHW700-NX or L685,458.

In yet another aspect, the invention provides pharmaceutical compositions for inhibiting or preventing malaria comprising an inhibitor of a Plasmodium falciparum signal peptide peptidase as disclosed herein and an antibody or antigen-binding fragment thereof as described herein that is immunologically specific for a Plasmodium falciparum signal peptide peptidase as disclosed herein, and at least one pharmaceutically acceptable carrier, diluent, and excipient. In particular embodiments, the inhibitor is (Z-LL)2-ketone, LY411575, NVP-AHW700-NX or L685,458.

In another aspect, the invention provides kits for treating or preventing malaria comprising a pharmaceutical composition as described herein and, optionally, instructions for use. In a further aspect, the invention provides kits for detecting the presence of a Plasmodium pathogen in a sample comprising an antibody of the invention immunologically specific for a Plasmodium falciparum signal peptide peptidase as disclosed herein and, optionally, instructions for use.

In still another aspect, the invention provides methods of screening for a compound that inhibits Plasmodium falciparum signal peptide peptidase (PfSPP) activity comprising the steps of contacting a Plasmodium falciparum signal peptide peptidase as disclosed herein, or membrane preparations comprising said polypeptide, with a test compound and a substrate that is converted by the PfSPP activity, wherein a decrease in the levels of substrate conversion as compared to control indicates that the compound is an inhibitor of the PfSPP activity. Suitable peptides or polypeptides for use in this aspect as PfSPP substrate include without limitation synthetic bovine prolactin signal peptide (Prl: EQKLISEEDLMDSKGSSQKGSRLLLLLVVSNLLLCQGVVS, SEQ ID NO:35; Prl-PP: EQKLISEEDLMDSKGSSQKGSRLLLLLVVSNLLLCQGPPS, SEQ ID NO:36, the underlined sequence is a Myc epitope tag). See Sato et al., 2006, Biochemistry 45(28):8649-56.

In a further aspect, the invention provides methods of detecting or quantifying PfSPP protein in a sample comprising the steps of: (a) contacting the sample with a PfSPP-specific antibody as described herein; and (b) detecting binding of the PfSPP protein in the sample to the antibody.

In a further aspect, the invention provides methods of detecting a Plasmodium parasite in a sample by detecting a Plasmodium signal peptide peptidase (SPP) protein in the sample, comprising the steps of: (a) contacting the sample with a PfSPP-specific antibody of the invention; and (b) detecting the binding of the Plasmodium SPP protein in the sample to the antibody, wherein binding of the Plasmodium SPP protein to the antibody indicates that the Plasmodium parasite is in the sample. In certain particular embodiments, the Plasmodium parasite is Plasmodium falciparum.

In another aspect, the invention provides methods of diagnosing Plasmodium infection in a human comprising the steps of: (a) contacting a sample obtained from the human with a PfSPP-specific antibody as described herein; and (b) assaying the sample for a Plasmodium signal peptide peptidase (SPP) polypeptide binding to the antibody, wherein binding of the Plasmodium SPP to the antibody indicates Plasmodium infection in the human. In certain particular embodiments, the Plasmodium is Plasmodium falciparum.

In another aspect, the invention provides kits for diagnosing Plasmodium infection in a human comprising a PfSPP-specific antibody of the invention and, optionally, instructions for use.

In yet another aspect, the invention provides malaria vaccines comprising a Plasmodium falciparum signal peptide peptidase as disclosed herein or an antigenic fragment thereof and a pharmaceutical carrier, diluent or excipient. In certain embodiments, the antigenic fragment comprises amino acid residues 246-264 of the sequence as identified by SEQ ID NO:2.

In another aspect, the invention provides compositions comprising a Plasmodium falciparum signal peptide peptidase as disclosed herein or an antigenic fragment thereof and a pharmaceutical carrier, diluent, or excipient. In certain embodiments, the antigenic fragment comprises amino acid residues 246-264 of the sequence as identified by SEQ ID NO:2. In other embodiments, the compositions are malaria vaccines.

In yet another aspect, the invention provides methods of immunizing a human in need thereof against Plasmodium infection or malaria comprising the step of administering a malaria vaccine as described herein to the human. In certain embodiments, the Plasmodium is Plasmodium falciparum.

Specific embodiments of the present invention will become evident from the following more detailed description of certain preferred embodiments and the claims.

### BRIEF DESCRIPTION OF THE FIGURES

FIG. 1A shows alignment of PfSPP from six strains of P. falciparum using ClustaIW2. One of the 3D7 sequences was taken from the Plasmodium database (PlasmoDB)(Gene ID: PF14_{—}0543). FIG. 1B shows alignment of malaria SPP from four Plasmodium species. “*” complete match; “:” conservative substitution; and “.” semi-conservative substitutions. Neither conservative nor semi-conservative substitutions affect the protein function. FIG. 1C shows a topology model of PfSPP by ConPred II. The following topological features of the protein are illustrated (by amino acid sequence residues): signal-anchor sequence (19-38, SEQ ID NO:23); two active site motifs YD (227-228) and LGLGD (265-269, SEQ ID NO:24); and the PALL (341-344) motif are indicated. Transmembrane (TM) regions are: TM1, 21-37 (SEQ ID NO:25); TM2, 39-55 (SEQ ID NO:26); TM3, 83-103 (SEQ ID NO:27); TM4, 113-133 (SEQ ID NO:28); TM5, 169-189 (SEQ ID NO:29); TM6, 196-216 (SEQ ID NO:30); TM7, 220-240 (SEQ ID NO:31); TM8, 263-283 (SEQ ID NO:32); TM9, 314-334 (SEQ ID NO:33); and TM10, 341-361 (SEQ ID NO:34). The region encoded by the cDNA insert in the yeast two-hybrid system screening assays is 183-412 (SEQ ID NO:6). The regions used to generate anti-PfSPP/ER peptide antibodies and recombinant maltose binding protein (MBP)-fusion protein are amino acid residues 246-264 (SEQ ID NO:4) and 226-266 (SEQ ID NO:3), respectively. The region used to generate anti-PfSPP C-terminus peptide antibodies are amino acid residues 393-412 (SEQ ID NO:5).

FIG. 2A depicts a phylogenetic tree of Plasmodium SPP. FIG. 2B indicates RNA expression of PfSPP-3D7 as a percentage of all other Plasmodium gene expression in the PlasmoDB. ER, Early Rings; LR, Late Rings; ET, Early Trophozoites; LT, Late Trophozoites; ES, Early Schizonts; LS, Late Schizonts; M, Merozoites; S, Sporozoites; and G, Gametozoites. Sorbitol- sorbitol-induced cell lysis; temperature-temperature cycling-induced cell lysis (see Doolan, D. L., MALARIA METHODS AND PROTOCOLS in METHODS IN MOLECULAR MEDICINE, Haynes and Moch (eds.) 2002, Humana Press).

FIG. 3 shows results of (A) Coomassie blue-stained electrophoretic gel assay showing the affinity-purified recombinant proteins MBP (lane 1) and MBP-PfSPP (lane 2); (B) Characterization of anti-PfSPP antibodies (Abs), showing an immunoblot illustrating that mono-specific anti-PfSPP polyclonal Abs reacted specifically to the recombinant MBP-PfSPP/ER (lanes 1 and 2) and native P. falciparum PfSPP (lanes 4 and 6). Pre-immune controls are shown in lanes 3 and 5. Lane 1, MBP; lanes 2 and 3, MBP-PfSPP/ER; lanes 4 and 5, P. falciparum extract; and lane 6, human RBC ghosts; and (C) Characterization of the affinity purified anti-PfSPP C-terminal pAb, showing an immunoblot illustrating that mono-specific anti-PfSPP (393-412) pAb reacted specifically to the recombinant MBP-PfSPP (lane 2) and native P. falciparum PfSPP (lane 3), but not with MBP alone (lane 1) and human RBC ghosts (lane 4). Lane 1, MBP; lane 2, MBP-PfSPP; lane 3, P. falciparum extract; lane 4, human RBC ghosts.

FIG. 4 depicts microphotographs of immunofluorescence microscopy images using specific antibodies, showing that PfSPP co-localized with EBA-175 (a microneme protein), but not with RAP 1 (a rhoptry marker) nor MSP 1 (merozoite surface protein) in the P. falciparum (3D7) schizonts.

FIG. 5 shows microphotographs of electron microscope images of immunogold-stained PfSPP in P. falciparum merozoites. (A) The pre-immune control showed no specific labeling. (B) Anti-PfSPP Abs showed specific labeling of the merozoite with gold particles in the micronemes (arrows) and the apical surface area (arrowheads). The parasite nucleus (Nu), rhoptries (Rh), and hemozoin (Hz) are indicated.

FIG. 6 shows (A) the Plasmodium SPP exofacial loop sequence distances between SPP from P. falciparum (1), P. vivax (2), P. berghei (3), and P. knowlesi (4), of which P. knowlesi infects primates and likely infect humans, whereas P. berghei does not infect humans; and (B) the presence of antibodies against PfSPP exofacial loop in patient plasma samples. Samples 1-10 were obtained from malaria patient plasma; samples 11-12 were obtained from two donors never exposed to malaria; and sample 13 was obtained from rabbit anti-PfSPP/ER serum that served as positive control.

FIG. 7A depicts a graph demonstrating PfSPP antibody-dependent inhibition of P. falciparum invasion of human RBCs. PfSPP/ER-specific antibodies () or pre-immune IgG (▪) were present in the culture medium at the time of invasion. FIG. 7B shows a photograph of an immunoblot using anti-PfSPP/ER Abs in preparations of P. falciparum culture supernatant separated at 40,000 g for 15 min (lane 1) and 12,000 g for 20 min (lane 2). Lane 3, pellet from 40,000 g centrifugation; lane 4, pellet from 12,000 g centrifugation. Equivalent amounts of supernatant samples (lanes 1 and 2) and pellet samples (lanes 3 and 4) were loaded. FIG. 7C shows photographs of immunoblots depicting RBC binding assays in suspension using the culture supernatant prepared by 12,000 g centrifugation. Normal (untreated, lane 1), trypsin-treated (lane 4), chymotrypsin-treated (lane 5), and neuraminidase-treated (lane 6) intact human RBC samples were analyzed by immunoblotting using anti-PfSPP/ER Abs following the binding assay. Soluble GST-5ABC (lane 3) was added to the binding assay to block binding of native PfSPP to normal RBCs. GST served as a negative control in lane 2. FIG. 7D shows a photograph of an immunoblot using anti-PfSPP/ER Abs and demonstrating specific-binding of native PfSPP to recombinant 5ABC domain. Lane 1, P. falciparum protein extract prepared using TX-100 (PE); lane 2, PE+GST-5ABC conjugated to beads; lane 3, PE+GST conjugated to beads; lane 4, GST-5ABC conjugated to beads only. FIG. 7E shows a photograph of an immunoblot assay using anti-His Abs depicting specific-binding of PfSPP and 5ABC in solution. Lane 1, MBP-PfSPP/ER conjugated to beads+Trx-5ABC; lane 2, MBP only conjugated to beads+Trx-5ABC; lane 3, MBP-PfSPP/ER conjugated to beads+Trx.

FIG. 8A shows chemical structures for certain signal peptide peptidase inhibitors. FIG. 8B presents a bar graph showing inhibition of RBC invasion by P. falciparum at the schizont stage treated with various SPP inhibitors for 20 h. FIG. 8C shows microphotographs of Giemsa-stained smears of RBC invaded by P. falciparum after 20 h of inhibitor treatment at 10 μM.

FIG. 9A shows microphotographs of blood smears of ring-stage parasites made in the presence of SPP inhibitors at 10 μM in 0.2% DMSO. FIG. 9B depicts a graph indicating inhibition of parasite growth as a result of (Z-LL)₂-ketone (▪), L-685,458 (•), and DAPT (♦) treatment. The IC₅₀ values of (Z-LL)₂-ketone and L-685,458 for live parasites were 984.9 nM and 173.5 nM, respectively. FIG. 9C is a schematic diagraph showing disruption of the P. falciparum (3D7 strain) PfSPP gene. PfSPP5′ and PfSPP3′ represent 5′ translated region (616 bp) and 3′ translated region (711 bp) of the PfSPP gene, respectively. Dashed lines represent bacterial vector sequences. Bold lines represent untranslated regions of the target PfSPP gene.

FIG. 10 shows the chemical structures of three signal peptide peptidase inhibitors NVP-AHW700-NX, LY411575 and LY450139.

### DETAILED DESCRIPTION OF THE INVENTION

All publications, patents and published patent applications cited herein are hereby expressly incorporated by reference for all purposes. Within this application, unless otherwise stated, the techniques utilized can be found in any of several well-known references such as: Molecular Cloning: A Laboratory Manual (Sambrook et al., 2001, Cold Spring Harbor Laboratory Press).

As used herein, the singular forms “a”, “an” and “the” include plural referents unless the context clearly dictates otherwise. For example, reference to a polynucleotide encompasses multiple polynucleotides.

This invention provides methods and reagents for inhibiting infection and replication of a Plasmodium parasite, such as Plasmodium falciparum, in a cell. Additionally, the invention provides methods and reagents for inhibiting Plasmodium invasion of a cell. Specifically, the invention provides polynucleotide and protein sequences for Plasmodium falciparum signal peptide peptidase (PfSPP), methods and reagents for inhibiting Plasmodium replication, growth or development in a cell, and vaccines that prevent malaria infection.

As used herein, the term “Plasmodium parasite” or “Plasmodium pathogen” refers to all strains of Plasmodium falciparum and their closely related Plasmodium species that infect humans, including without limitation Plasmodium vivax, Plasmodium malariae, and Plasmodium ovale.

In certain aspects, the invention provides nucleic acids and polypeptides of PfSPP and the polynucleotide and amino acid sequences thereof. In one aspect, the invention provides isolated nucleic acids comprising a polynucleotide sequence (a) that is identified by SEQ ID NO:1; (b) that encodes a polypeptide comprising the amino acid sequence as identified by SEQ ID NO:2, or (c) that is complementary to the nucleotide sequence of (a) or (b).

In another aspect, the invention provides a vector comprising a nucleic acid of the invention. In certain embodiments, the invention provides an expression vector comprising the nucleic acids provided by the invention. In certain particular embodiments, nucleic acids provided by the invention are operably linked to gene expression regulatory element such as, inter alia, a promoter sequence, and optionally to an enhancer sequence, in the expression vector. It is understood by one skilled in the art that the expression vector comprises the nucleic acid of the invention in an orientation that the PfSPP gene can be expressed and transcribed from the promoter.

The term “vector” as used herein refers to any molecule (e.g., nucleic acid, plasmid, or virus) used to transfer coding information to a host cell. The term “expression vector” refers to a vector that is suitable for transformation of a host cell and contains nucleic acid sequences that direct and/or control the expression of inserted heterologous nucleic acid sequences. Expression includes without limitation processes such as transcription, translation, and RNA splicing, if introns are present. In another aspect, the invention provides host cells that comprise and express the expression vectors of the invention.

The term “operably linked” is used to refer to an arrangement wherein nucleic acids that are operably linked are arranged so that each of the nucleic acids performs its intended and usual function. For example, a promoter is operably linked to a nucleic acid encoding a protein is the promoter and the coding sequence are covalently linked in an arrangement wherein the promoter directed production of RNA from the portion of the nucleic acid encompassing the coding sequence. In certain embodiments, promoter and other elements involved in regulating transcription of portions of vector or other nucleic acid encoding a protein are present in flanking sequences, wherein the flanking sequences are located 5′ or 3′ to the beginning of the portion of the nucleic acid encoding a polypeptide. Elements in flanking sequences operably linked to a coding sequence are capable of effecting the replication, transcription and/or translation of the coding sequence. A flanking sequence need not be contiguous with the coding sequence, so long as it functions correctly.

The genome of Plasmodium falciparum (strain 3D7) has been sequenced and one putative signal peptide peptidase (SPP) was identified (NP_{—}702432). See Gardner et al., 2002, Nature 419:498-511. Signal peptide peptidases are a class of aspartic intramembrane proteases that cleave type II transmembrane proteins. GenBank Accession No. XM_{—}001348681 provides a predicted cDNA sequence of the PfSPP gene on chromosome 14 of Plasmodium falciparum strain 3D, and the presumptive amino acid sequence of PfSPP encoded therefrom. Nyborg et al. reported the production of a putative PfSPP protein from a synthesized cDNA according to NP_{—}702432 with the published amino acid sequence. Nyborg showed that the PfSPP so produced exhibited some protease activity to an SPP substrate in an in vitro assay.

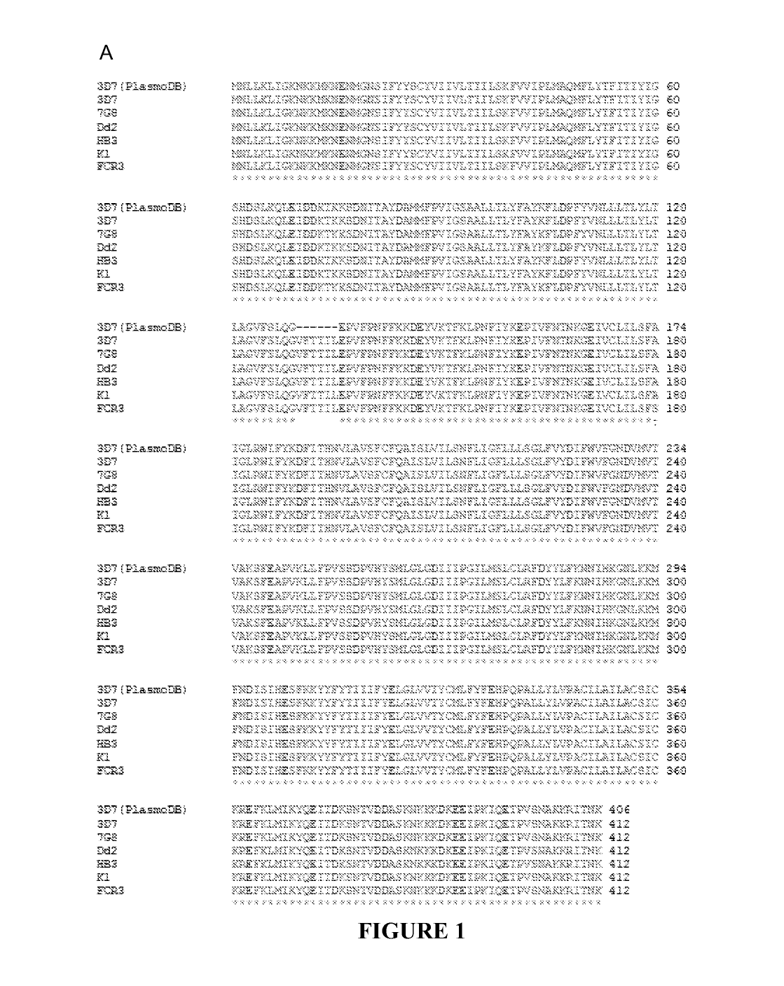

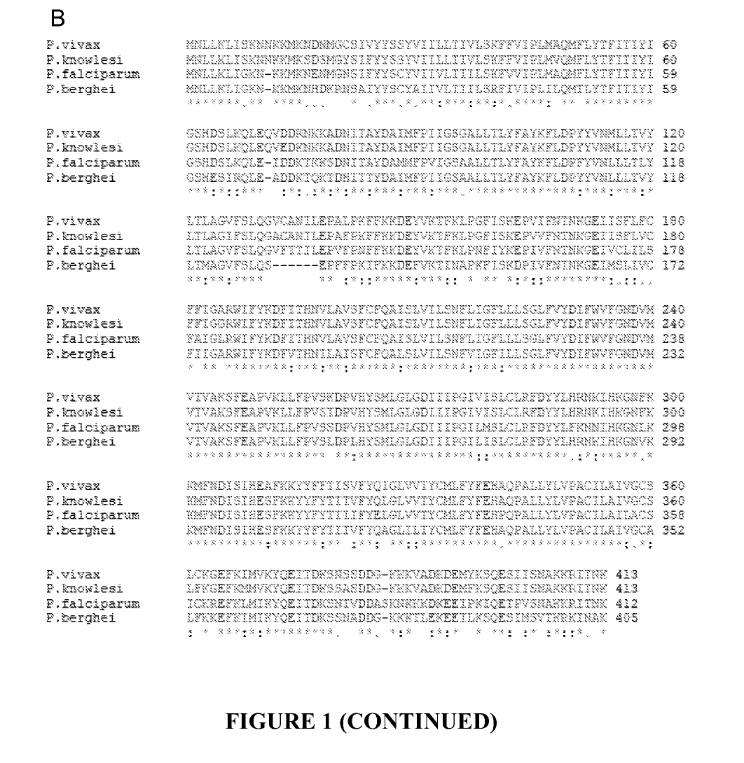

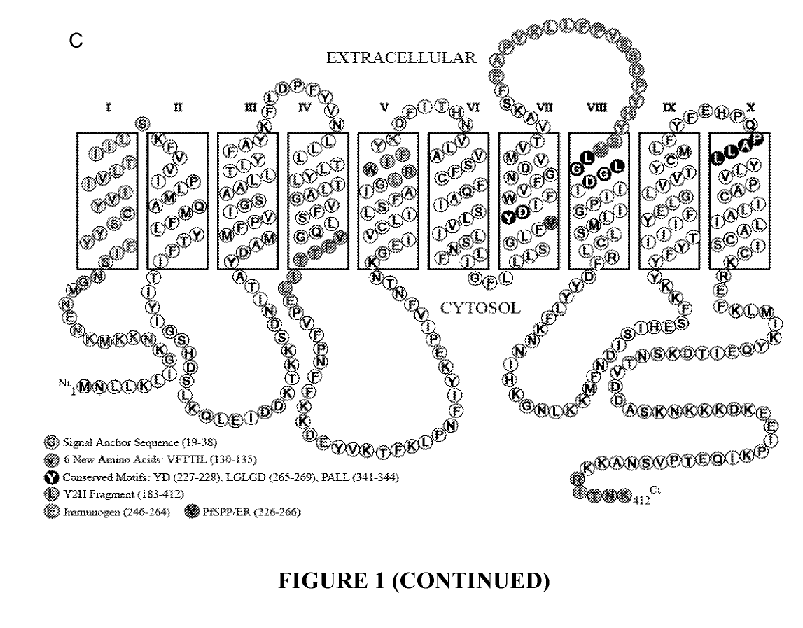

As disclosed herein, it was unexpectedly discovered that the predicted PfSPP cDNA structure and amino acid sequence under NP_{—}702432 were incorrect. The correct PfSPP protein sequence as revealed herein contains an additional 6 amino acid residues having the sequence VFTTIL between glycine 129 and glutamic acid 130 as set forth in the published sequence (compare NP_{—}702432 with FIG. 1A). The differences in the amino acid sequence may have been due to an incorrect prediction of the cDNA structure of the PfSPP gene in the PlasmoDB, wherein a sequence of 18 nucleotides encoding the VFTTIL amino acid sequence were assigned to the 4^{th} intron incorrectly, rather than being properly assigned to the 4^{th} exon of the PfSPP gene. Thus the skilled worker, as illustrated by Nyborg et al., mistakenly thought that the PfSPP sequence was 6 amino acids shorter than it actually is. It is appreciated in the art that deletions in a protein, especially a transmembrane protein, can alter the conformation and the topology of the protein on the membrane, and thus can affect the activity of the protein. It is noted that among the four Plasmodium species shown in FIG. 1B, three species, P. falciparum, P. vivax, and P. knowlesi, which infect humans or primates, all contain the additional 6-amino acid peptide between the glycine and glutamic acid residues (amino acid position 129 and 130, respectively, according to the sequence set forth in PlasmoDB). The term “PfSPP” as used herein refers to a polypeptide comprising the amino acid sequence identified by SEQ ID NO:2 (unless indicated otherwise), i.e. containing the 6 amino acids correctly disclosed as being part of the PfSPP protein herein.

In yet another aspect, the invention provides a host cell comprising the vector or expression vector of the invention. The term “host cell” as used herein refers to a cell that has been transformed, or is capable of being transformed with a nucleic acid encoding a polypeptide and then of expressing the polypeptide encoded therein. Suitable host cells include without limitation mammalian cells, bacteria cells, a yeast cells or insect cells.

In another aspect, the invention provides membrane preparations comprising a polypeptide comprising the amino acid sequence as identified by SEQ ID NO:2. Because of the membrane-bound properties of the protein, the PfSPP protein is isolated from the parasite or parasite infected cells in a membrane preparation or similar environment to preserve the proper secondary, tertiary and/or quaternary structure of the protein. The term “a membrane preparation” as used herein includes crude membrane preparations or membranes purified using methods well known in the art, so long as the PfSPP protein maintains its functional conformation. In addition, a membrane preparation can also refer to a detergent-solubilized environment, i.e., the transmembrane protein PfSPP can maintain its activity and functional conformation as a detergent-solubilized form. In certain embodiments, the detergent-solubilized Plasmodium SPP is solubilized by 1% Triton X-100. In certain other embodiments, the detergent-solubilized Plasmodium SPP is solubilized by 1% n-dodecyl beta-D-maltoside (DDM).

As used herein, the twenty conventional amino acids and their abbreviations follow conventional usage. See IMMUNOLOGY—A SYNTHESIS, 2nd Edition, (Golub and Gren, Eds.), Sinauer Associates: Sunderland, Mass., 1991, incorporated herein by reference for any purpose. Stereoisomers (e.g., D-amino acids) of the twenty conventional amino acids; unnatural amino acids such as α-, α-di-substituted amino acids, N-alkyl amino acids, and other unconventional amino acids can also be suitable components for polypeptides of the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues can be divided into classes based on common side chain properties:

1) hydrophobic: norleucine (Nor), Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Tip, Tyr, Phe.

Conservative amino acid substitutions can involve exchange of a member of one of these classes with another member of the same class. Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions can involve the exchange of a member of one of these classes for a member from another class. Such substituted residues can be introduced into regions of the human antibody that are homologous with non-human antibodies, or into the non-homologous regions of the molecule.

In making such changes, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. One can also identify epitopes from primary amino acid sequences on the basis of hydrophilicity. These regions are also referred to as “epitopic core regions.”

Exemplary amino acid substitutions are set forth in Table 1.

TABLE 1
Amino Acid Substitutions

| | | Preferred |
|---|---|---|
| Original Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, Val, | Ile |
| | Met, Ala, Phe | |
| Lys | Arg, 1,4 Diamino- | Arg |
| | butyric cid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |
| | Norleucine | |

A skilled artisan will be able to determine suitable conservative amino acid substitutions of the polypeptide as set forth herein using well-known techniques. One skilled in the art can identify suitable areas of the molecule that can be changed without destroying activity by targeting regions not believed to be important for activity as indicated by the sequence alignments shown in FIG. 1. The skilled artisan can identify residues and portions of the molecules that are conserved among similar polypeptides. It is understood that even areas that can be important for biological activity or for structure can be subject to conservative amino acid substitutions without destroying the biological activity or without adversely affecting the polypeptide structure.

In another aspect, the invention provides purified antibodies or antigen-binding fragments thereof that specifically bind to PfSPP of the invention. In particular embodiments, antibodies or antigen-binding fragments thereof of this invention recognize an epitope located within amino acid residues 246-264 of SEQ ID NO:2 (SEQ ID NO:4) or within amino acid residues 393-412 of SEQ ID NO: 2 (SEQ ID NO:5).

The term “specifically bind,” “specifically bound,” or “specific binding,” when used in the context of antibody-antigen interaction, is consistent with the usage in the field of immunology. For example, an antibody specifically binds to an antigen when the binding occurs under stringent binding conditions as defined in the art of immunology. The binding of the antibody to a non-antigen, or a molecule that does not have the epitope is insubstantial or undetectable using detection methods commonly used in the art of immunology. Specific binding can also be determined by competition; for example, specific binding between an antibody and the antigen under stringent binding conditions can be competed by the same antibody or antigen, but not by other unrelated molecules.

The antibodies provided by the invention can be raised, using methods well known in the art, in animals by inoculation the animals with cells that express the full-length PfSPP protein of this invention or antigenic fragments thereof, or cell membrane preparations from such cells, whether crude membrane preparations or membranes purified using methods well known in the art. Alternatively or additionally, animals can be inoculated with purified preparations of proteins, including fusion proteins comprising PfSPP, particularly fusion proteins comprising fragments of the PfSPP protein of the invention fused to heterologous proteins and expressed using genetic engineering means in bacterial, yeast or eukaryotic cells. Suitable antigen peptides include without limitation peptides comprising PfSPP amino acid residues 226-266 (SEQ ID NO:3), 246-264 (SEQ ID NO:4), 393-412 (SEQ ID NO:5), or 183-412 (SEQ ID NO:6), or fragments thereof, of the amino acid sequence identified by SEQ ID NO:2. In certain particular embodiments, the antigenic peptide comprises amino acid residues 246-264 (SEQ ID NO:4) or 393-412 (SEQ ID NO:5) of the amino acid sequence identified by SEQ ID NO:2.

Fusion proteins can be isolated from such cells to varying degrees of homogeneity using conventional biochemical methods. Heterologous proteins can optionally be cleaved by a desirable protease at a protease cleavage site engineered into the fusion protein, so that cleavage can be effected before the PfSPP proteins or fragments are used as immunogens. Synthetic peptides made using established synthetic methods in vitro are also contemplated as immunogens to produce the antibodies of the invention. Animals that are useful for such inoculations include cows, sheep, pigs, chickens, mice, rats, rabbits, hamsters, goats and primates. Preferred animals for inoculation are rodents (including mice, rats, hamsters) and rabbits. The most preferred animal for making polyclonal antibodies is the rabbit.

In certain embodiments, the invention provides purified antibodies or antigen-binding fragments thereof that recognize an epitope located within amino acid residues 246-264 of SEQ ID NO: 2. In certain other embodiments, the invention provides purified antibodies or antigen-binding fragments thereof that recognize an epitope located within amino acid residues 393-412 of SEQ ID NO: 2. In certain further embodiments, the antigen-binding fragment comprises a variable region fragment; in certain other embodiments, the antigen-binding fragment is a Fab or F(ab′)₂ fragment. Fragments are produced by any number of methods, including but not limited to proteolytic or chemical cleavage, chemical synthesis or preparation of such fragments by means of genetic engineering technology (See, for example, Andrew et al., 1992, “Fragmentation of Immunoglobulins” in CURRENT PROTOCOL IN IMMUNOLOGY, Unit 2.8, Greene Publishing Assoc. and John Wiley & Sons). The present invention also encompasses single-chain antibodies that are specific for PfSPP of the invention, made by methods known to those of skill in the art (U.S. Pat. No. 4,946,778; Bird, 1988, Science 242:423-42; Huston et al., 1988, Proc. Natl. Acad. Sci. USA 85:5879 5883; and Ward et al., 1989, Nature 334:544-54).

In accordance with this aspect, the invention provides Plasmodium neutralizing antibodies or antigen-binding fragments thereof that neutralize or inhibit infection by a Plasmodium parasite. In certain particular embodiments, the Plasmodium parasite is Plasmodium falciparum.

In certain particular embodiments of this aspect, antibodies of this invention are monoclonal antibodies or antigen-binding fragments thereof. Such antibodies are made using methods and techniques well known to those of skill in the art. Monoclonal antibodies provided by the present invention can be produced by hybridoma cell lines that can be made by methods well known in the art and as described herein.

Monoclonal antibodies immunologically-reactive against antigenic peptides of the invention can be prepared according to well-known techniques, such as those exemplified in U.S. Pat. No. 4,196,265, incorporated herein by reference. Hybridomas producing monoclonal antibodies against the antigenic peptides of the invention are produced by well-known techniques. Usually, the process involves inter alia fusing an immortalized cell line with a B-lymphocyte that produces the desired antibody. Immortalized cell lines are usually neoplastically-transformed mammalian cells, particularly myeloma cells of rodent, bovine, and human origin. Rodents such as mice and rats are preferred animals, however, the use of rabbit or sheep cells is also possible. Mice are preferred, with the BALB/c mouse being most preferred as this is most routinely used and generally gives a higher percentage of stable fusions.

Techniques for obtaining antibody-producing lymphocytes from mammals injected with antigens are well known. Generally, peripheral blood lymphocytes (PBLs) are used if cells of human origin are employed, or spleen or lymph node cells are used from non-human mammalian sources. A host animal is injected with repeated dosages of the purified antigen, and the animal is permitted to generate the desired antibody-producing cells before they are harvested for fusion with the immortalizing cell line. Most frequently, immortalized cell lines are rat or mouse myeloma cell lines that are employed as a matter of convenience and availability. Techniques for fusion are also well known in the art, and in general involve mixing the cells with a fusing agent, such as polyethylene glycol.

Generally, following immunization somatic cells with the potential for producing antibodies, specifically B-lymphocytes (B-cells), are selected for use in producing monoclonal antibodies (mAb). These cells can be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately fifty million to two hundred million lymphocytes.

Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells can be used, as are known to those of skill in the art. Available murine myeloma lines, such as those from the American Type Culture Collection (ATCC), Manassas, Va. 20110-2209, USA, can be used in the hybridization. For example, where the immunized animal is a mouse, cell lines identified as P3-X63/Ag8, X63-Ag8.653, NS1/1.Ag 4 1, Sp210-Ag14, FO, NSO/U, MPC-11, MPC11-X45-GTG 1.7 and S194/5XX0 Bul can be used. For rats, cell lines identified as R210.RCY3, Y3-Ag 1.2.3, IR983F and 4B210 can be used; and human cell lines U-266, GM1500-GRG2, LICR-LON-HMy2 and UC729-6 are all useful in connection with human cell fusions. One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that can be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio can vary from about 20:1 to about 1:1, respectively, in the presence of an agent or agents (chemical or electrical) that promote fusion of cell membranes. Fusion methods using Sendai virus have been described (Kohler et al., 1975, Nature 256:495; Kohler et al., 1976, Eur. J. Immunol. 6:511; Kohler et al., 1976, Eur. J. Immunol. 6:292), and those using polyethylene glycol (PEG), such as 37% (v/v) PEG, by Gefter et al., (1977). The use of electrically induced fusion methods can also be appropriate (Goding, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks de novo synthesis of nucleotides in cell culture media. Exemplary and preferred agents are aminopterin, methotrexate, and azaserine, where aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. The preferred selection medium is HAT (Köhler et al., 1975, Nature 256:495.). Myeloma cells can also be defective in key enzymes of a nucleotide salvage pathway, e.g., hypoxanthine phosphoribosyl transferase (HPRT). B-cells, typically having a wild-type phenotype for enzymes of said nucleotide salvage pathway are viable in unsupplemented media but have a limited life span in culture and generally die within about two weeks. Therefore, the only cells that can survive in the selective media are those hybrids formed from myeloma and B-cells.

Culturing lymphocyte/myeloma cell fusion products under these conditions provides a population of hybridomas from which specific hybridomas are selected. Typically, selection of hybridomas is performed by culturing cells from single-clone dilution in microtiter plates, followed by testing individual clonal supernatants (after about two to three weeks) for the desired reactivity. Hybridomas secreting the desired antibody are selected using standard immunoassays, such as Western blotting, ELISA (enzyme-linked immunosorbent assay), RIA (radioimmunoassay), or the like. Antibodies are recovered from the medium using standard protein purification techniques (such as Tijssen, 1985, Id.). The assay should be sensitive, simple and rapid, such as radioimmunoassay, enzyme immunoassays, cytotoxicity assays, plaque assays, dot immunobinding assays, and the like.

The selected hybridomas are then serially diluted and cloned into individual antibody-producing cell lines, which clones can then be propagated indefinitely to provide mAbs. The cell lines can be exploited for mAb production in at least two ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means can be further purified, if desired, using filtration, centrifugation and various chromatographic methods such as HPLC or affinity chromatography.

Many references are available to provide guidance in applying the above techniques, including Kohler et al. (1980, HYBRIDOMA TECHNIQUES, Cold Spring Harbor Laboratory, New York); Tijssen (1985, Id.); Campbell (1984, MONOCLONAL ANTIBODY TECHNOLOGY, Elsevier: Amsterdam); Hurrell (1982, Id.). Monoclonal antibodies can also be produced using well known phage library systems. See, for example, Huse et al. (1989, Science 246:1275); Ward et al. (1989, Nature 341:544).

In certain other embodiments of this aspect, the antibody is a humanized, human, chimeric, or CDR-grafted antibody, or an antigen-binding fragment thereof. The invention also includes chimeric antibodies, comprised of light chain and heavy chain peptides specific for PfSPP-derived epitopes described herein. The chimeric antibodies embodied in the present invention include those that are derived from naturally occurring antibodies as well as chimeric antibodies made by means of genetic engineering technology well known to those of skill in the art.

Humanized antibodies are antibody molecules from non-human species that bind the desired antigen having one or more complementarity determining regions (CDRs) from the non-human species and a framework region from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, for example, U.S. Pat. No. 5,585,089, and Riechmann et al., 1988, Nature 332:323, which are incorporated herein by reference in their entireties.) Antibodies can be humanized using a variety of techniques known in the art including, for example, CR-grafting (European Patent Application, Publication No. EP239400; PCT publication No. WO 91/09967; U.S. Pat. Nos. 5,225,539; 5,530,101; and 5,585,089), veneering or resurfacing (European Patent Applications, Publication Nos. EP592106; EP519596; Padlan, 1991, Molecular Immunology 28:489 498; Studnicka et al., 1994, Protein Engineering 7: 805 814; Roguska et al., 1994, Proc. Natl. Acad. Sci. USA 91:969-973), and chain shuffling (U.S. Pat. No. 5,565,332).

Completely humanized antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications Nos. WO 98/46645, WO 98/50433, WO 98/24893, WO 98/16654, WO 96/34096, WO 96/33735, and WO 91/10741; each of which is incorporated herein by reference in its entirety. Completely humanized antibodies which recognize a selected epitope can be generated using a technique referred to as “guided selection.” In this approach a selected non-human monoclonal antibody, e.g., a mouse antibody, is used to guide the selection of a completely humanized antibody recognizing the same epitope. (Jespers et al., 1988, Biotechnology 12:899-903).

In addition, techniques developed for the production of “chimeric antibodies” (Morrison et al., 1984, Proc. Natl. Acad. Sci. USA 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, 1988, Science 242:423-42; Huston et al., 1988, Proc. Natl. Acad. Sci. USA 85:5879 5883; and Ward et al., 1989, Nature 334:544-54) can be adapted to produce single chain antibodies immunologically-reactive to a P. falciparum signal peptide peptidase of the invention. Single chain antibodies are formed by linking the heavy and light chain fragments of the Fᵥ region via an amino acid bridge, resulting in a single chain polypeptide. Techniques for the assembly of functional Fᵥ fragments in E. coli can also be used (Skerra et al., 1988, Science 242:1038 1041).

In a further aspect, the invention provides methods of detecting or quantifying PfSPP protein in a sample comprising the steps of (a) contacting the sample with the antibody of the invention, and (b) detecting the binding of the PfSPP protein in the sample to the antibody. In yet another aspect, the invention provides methods of detecting a Plasmodium parasite in a sample by detecting a Plasmodium SPP protein in the sample comprising the steps of (a) contacting the sample with the antibody of the invention, and (b) detecting the binding of the Plasmodium SPP protein in the sample to the antibody, wherein the binding of the Plasmodium SPP protein to the antibody indicates that the Plasmodium parasite is in the sample.

A variety of assays are known to the skilled in the art for use to detect or quantify an antigen or an antibody in a sample. Exemplary assays are described in detail in, for example, ANTIBODIES: A LABORATORY MANUAL, Harlow and Lane (eds.), Cold Spring Harbor Laboratory Press, 1988. Representative examples of such assays include without limitation concurrent immunoelectrophoresis, radio-immunoassays, radio-immunoprecipitations, enzyme-linked immunosorbent assays (ELISA), dot blot assays, inhibition or competition assays, and sandwich assays.

In accordance with the methods described above, in further aspects of the invention, kits are provided for detecting or quantifying the PfSPP protein in a sample or kits for diagnosing Plasmodium infection in a human, comprising the PfSPP-specific antibodies of the invention and optionally instructions for use.

In yet another aspect, the invention provides methods of inhibiting a Plasmodium parasite infection or replication, growth or development in a cell comprising contacting the Plasmodium parasite with the PfSPP-specific antibody of the invention. In certain particular embodiments, the cell is an erythrocyte. In certain other embodiments, the antibody or antigen-binding fragment recognizes an epitope located within amino acid residues 246-264 of SEQ ID NO: 2. In yet other embodiments, the antibody or antigen-binding fragment thereof recognizes an epitope located within amino acid residues 393-412 of SEQ ID NO: 2.

In certain particular embodiments of this aspect, the inventive methods further comprise contacting the cell with an effective amount of a signal peptide peptidase inhibitor. Signal peptide peptidase (SPP) and presenilin 1 and 2 belong to a family of intramembrane cleaving aspartyl proteases that cleaves a variety of transmembrane substrates. SPP and presenilins all contain a conserved active site motif of YD and GXGD in adjacent transmembrane domains and a conserved PALL motif near the C-terminus of the proteins. Both conserved motifs have been shown to be crucial for protease activity (Martoglio et al., 2003, Hum. Mol. Genet. 12 Spec. No. 2, R201-R206; Xia et al., 2003, J. Cell Sci. 116:2839-44; Wang et al., 2004, Neurobiol. Dis. 15:654-66). SPP and presenilin, however, differ in several aspects. SPP and presenilin have inverted active site topologies: in SPP, the first catalytic motif YD is orientated from cytosol across the membrane to ER lumen or extra-cellular space, followed by the second catalytic motif GxGD, which is oriented from the ER lumen or extracellular space across the membrane to cytosol. The orientations of these two catalytic motifs in presenilin are reversed as compared with those in SPP (Friedmann et al. 2004, J. Biol. Chem. 279:50790-98). Further, presenilin requires three additional components to function (being recognized as γ-secretase expressed inter alia in mammalian neural tissues), whereas SPP activity can be reconstituted by expressing SPP alone. In addition, presenilin cleaves type I membrane proteins, and SPP cleaves type II membrane proteins, wherein types I and II are single pass transmembrane molecules, with the distinction that the type I transmembrane proteins have their N-terminal domains targeted to the ER lumen during synthesis (and the extracellular space, if mature forms are located on plasma membrane), while type II have their N-terminal domains targeted to the cytoplasm.

Several Plasmodium proteases have been identified. Using proteases as drug targets for malaria treatment, however, has not been successful because none of the previously identified proteases was found to be essential to Plasmodium survival. For example, intramembrane serine proteases, PfROM1 and PfROM4, were known to exist in the micronemes and merozoite surface (Baker et al., 2006, PLoS Pathog. 2:e-113; O'Donnell et al., 2006, J. Cell Biol. 174:1023-33). Despite extensive research, however, drug development efforts targeting these parasite proteases have not been successful because of the functional redundancy of the protease activities expressed thereby.

Additionally, Plasmodium aspartyl protease plasmepsins were identified as enzymes that cleave hemoglobin as source of amino acids for parasite growth in erythrocytes. Plasmepins are non-membrane bound aspartyl proteases, having similarities with the HIV protease. Attempts have been made to screen known HIV protease inhibitors for compounds that inhibit plasmepsins and reduce parasitemia. See Andrews et al. 2006, supra. Because of the structural differences between plasmepsins and SPP, however, the known HIV protease inhibitors are not likely suitable inhibitors for Plasmodium SPP. In addition, it has been recognized in the art that hemoglobin-degrading plasmepsins are not promising drug targets for treating malaria because blockade of plasmepsin activities was not found to be lethal to the parasite. See Liu et al., 2006, Proc. Natl. Acad. Sci. 103:8840-45.

It was surprisingly discovered as set forth in the instant application that Plasmodium SPP can be an effective drug target for malaria treatment because, inter alia, the single copy of the PfSPP gene in the P. falciparum genome was found to be essential to the survival of the parasite (See Example 10 herein). Thus, within the scope of the instant invention are methods of inhibiting Plasmodium infection and replication in a cell comprising the step of contacting the cell with a signal peptide peptidase inhibitor.

The role of Plasmodium SPP in the Plasmodium parasite life cycle was not previously known. It was also unexpectedly discovered by the applicants that the PfSPP protein was expressed in the micronemes and on the apical end of merozoites, both cellular compartments that are points of contact during initial parasite invasion. Thus, in another aspect, the invention provides methods of inhibiting a Plasmodium parasite invasion of a cell comprising contacting the Plasmodium parasite with a signal peptide peptidase inhibitor. In yet another aspect, the invention provides methods of inhibiting Plasmodium replication, growth or development in a cell comprising contacting the cell with a signal peptide peptidase inhibitor. In certain embodiments, the cell is an erythrocyte. In accordance with these aspects, certain embodiments provide methods that further comprise contacting the cell with the PfSPP-specific antibody of the invention or antigen-binding fragment thereof.

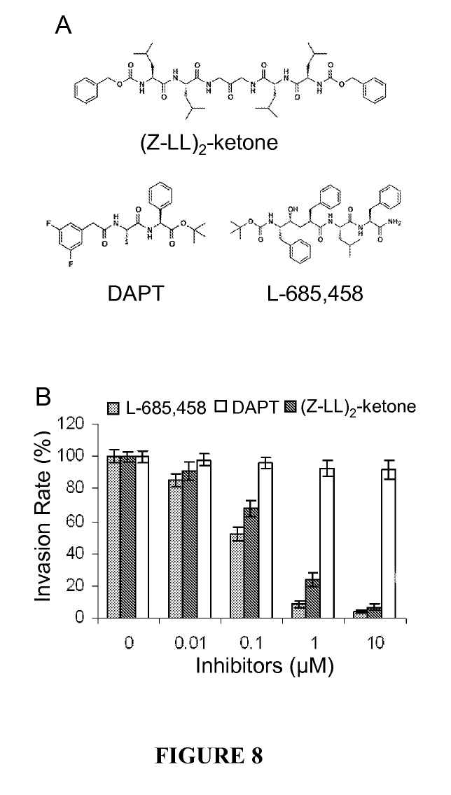

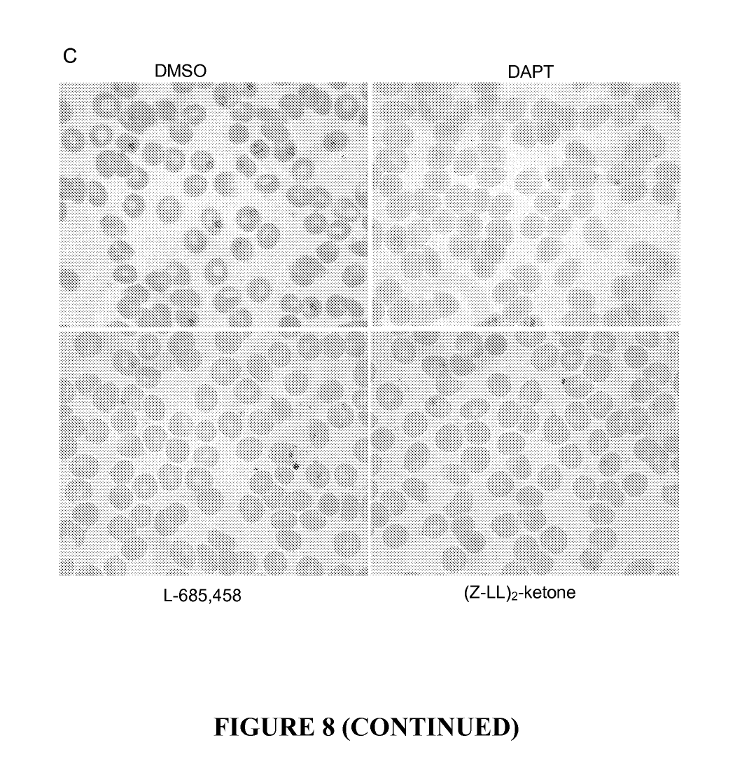

Well-characterized chemical libraries of presenilin inhibitors are available. Because of the structural features shared by presenilins and SPP, some presenilin or γ-secretase inhibitors initially identified against presenilin can also target SPP. As presenilins and SPPs possess opposite active site orientations, however, several inhibitors can be and have been synthesized and determined to have selective specificity for one or the other. For example, DAPT preferentially inhibits presenilins, whereas L-685,458 and (Z-LL)₂-ketone are more specific for the SPPs. Consistent with this specificity, L-685,458 and (Z-LL)₂-ketone efficiently inhibited merozoite invasion in human erythrocytes whereas the DAPT inhibitor had minimal effect (FIG. 8). It is within the skill of a synthetic organic chemist or a structural biochemist to modify the structure of presenilin inhibitors for compounds that have differential specificity for signal peptide peptidase, especially PfSPP. High levels of sequence identity and homology among PfSPP from different species indicate that a signal peptide peptidase inhibitor effective for PfSPP can be effective for the parasite SPP in other Plasmodium species as well.

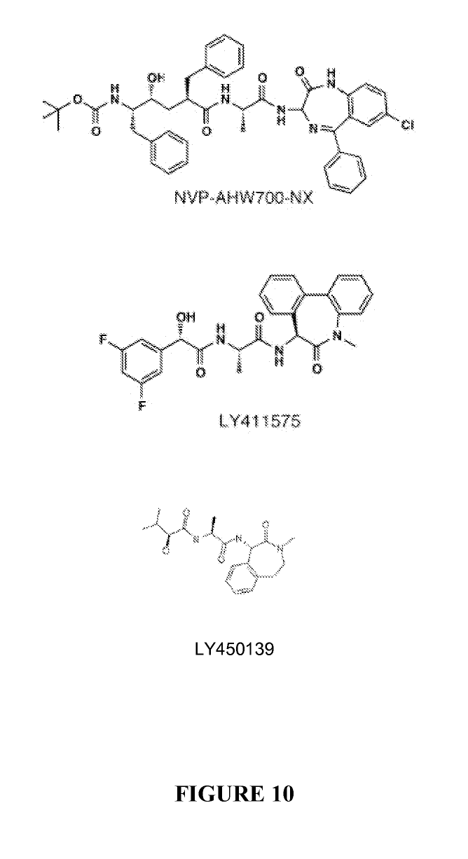

Suitable signal peptide peptidase inhibitors for use with the methods of the instant invention include without limitation L-685,458, LY411575 (a benzodiazepine/benzolactam analogue produced by Eli Lilly & Co.), NVP-AHW700-NX (a derivative of L-685,458), LY450139 and (Z-LL)₂-ketone. The structures of the compounds are shown in FIGS. 8 and 10. In certain particular embodiments, the signal peptide peptidase inhibitor is L-685,458 or (Z-LL)₂-ketone. It has been suggested in the art that analogues to the transition state mimicking gem-diol, such as L-685,458 and NVP-AHW700-NX can be effective SPP inhibitors (Weihofen et al., 2003, J. Biol. Chem. 278:16528-16533).

In another aspect, the invention provides methods of treating or preventing malaria in a human in need thereof comprising administering to the subject an effective amount of a signal peptide peptidase inhibitor. In certain embodiments of these aspects, the inventive methods further comprise administering to the human an effective amount of the PfSPP-specific antibody of the invention or antigen-binding fragment thereof.

In another aspect, the invention provides malaria vaccines comprising the PfSPP polypeptide of the invention or an antigenic fragment thereof and a pharmaceutical carrier, diluent or excipient. In certain embodiments, the antigenic fragment comprises amino acid residues 246-264 of the sequence as identified by SEQ ID NO:2. In certain other embodiments, the antigenic fragment comprises amino acid residues 393-412 of the sequence as identified by SEQ ID NO:2.

Malaria vaccine development has been hampered by parasite immune evasion as a result of antigen variation. The Plasmodium parasite expresses different antigens during different stages of the life cycle. Thus, vaccine-induced immune responses targeting a specific antigen expressed in one stage of the life cycle are ineffective at different stages of the parasite life cycle. The inventive vaccines based on PfSPP are advantageous because this antigen is expressed at multiple stages during infection.

Vaccines comprising effective amounts of the PfSPP polypeptides of the invention or antigenic fragments thereof induce immune responses in an immunized individual. The immune responses can lead to the production of antibodies that protect the vaccinated individual from Plasmodium infection. In certain embodiments, the Plasmodium infection is a Plasmodium falciparum infection. In certain embodiments, the immunogens can be linked to a carrier moiety, such as a carrier protein, in monomeric or multimeric form. Examples of carrier moieties include without limitation keyhole limpet hemocyanin (as described in U.S. Pat. No. 5,855,919), multiple antigen peptide (MAP) (as described in U.S. Pat. No. 5,229,490), tetanus toxoid, poly-L-(LYS:GLU), peanut agglutinin, poly-D-Lysine, diphtheria toxoid, ovalbumin, soybean agglutinin, bovine serum albumin (BSA), and human serum albumin. The vaccine composition can also include adjuvants, such as alum or squalene, and preservatives, such as thimerosal (thiomersal), phenoxyethanol, or formaldehyde.

The vaccines as described herein can also be administered in combination with other Plasmodium vaccines known in the art. For example, vaccines comprising fragments of Plasmodium falciparum merozoite surface protein-1 (MSP-1) (see U.S. Pat. Nos. 7,150,875, 7,256,281, and 7,306,806), P. falciparum apical membrane antigen 1 ectodomain (AMA-1/E) (see U.S. Pat. No. 7,060,276), or a polypeptide that constitutes a B cell epitope of the P. falciparum circumsporozoite (CS) protein (see U.S. Pat. No. 6,942,866), can provide added efficacy when administered together with a vaccine according to the present invention.

Vaccines of the invention can be administered by injection, such as intramuscularly or subcutaneously, orally by means of a tablet or an enteric capsule, as a suppository, as a nasal spray, or by any other suitable routes of administration. The dose of the vaccine depends on the route of administration and a number of other factors, including body weight, the chosen carrier, the adjuvant, and the total number of inoculations to be performed.

In a further aspect, the invention provides compositions comprising PfSPP-specific antibodies of the invention and optionally a signal peptide peptidase inhibitor. The invention also provides pharmaceutical compositions comprising PfSPP-specific antibodies of the invention and optionally a signal peptide peptidase inhibitor, and at least one excipient, diluent or carrier.

The pharmaceutical compositions of the invention can contain formulation materials such as pharmaceutically acceptable carriers, diluents, excipients for modifying, maintaining, or preserving, in a manner that does not hinder the activities of the therapeutic compounds or molecules described herein, for example, pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; Triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

The primary vehicle or carrier in a pharmaceutical composition can be either aqueous or non-aqueous in nature. For example, a suitable vehicle or carrier for injection can be physiological saline solution, or artificial cerebrospinal fluid. Optimal pharmaceutical compositions can be determined by a skilled artisan depending upon, for example, the intended route of administration, delivery format, desired dosage and recipient tissue. See, e.g., REMINGTON'S PHARMACEUTICAL SCIENCES, supra. Such compositions can influence the physical state, stability, and effectiveness of the composition.

The pharmaceutical composition to be used for in vivo administration typically is sterile and pyrogen-free. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical compositions of the invention have been formulated, they can be stored in sterile vials as a solution, suspension, gel, emulsion, solid, or as a dehydrated or lyophilized powder. Such formulations can be stored either in a ready-to-use form or in a form (e.g., lyophilized) that is reconstituted prior to administration.

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician can titer the dosage, using the in vitro effective doses provided herein as a reference, and modify the route of administration to obtain the optimal therapeutic effect.

The dosing frequency will depend upon the pharmacokinetic parameters of the therapeutic molecules in the formulation. For example, a clinician administers the composition until a dosage is reached that achieves the desired effect. The composition can therefore be administered as a single dose, or as two or more doses (which can or can not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, subcutaneous, or intralesional routes; by sustained release systems or by implantation devices. The pharmaceutical compositions can be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

Pharmaceutical compositions of the invention can be administered alone or in combination with other therapeutic agents, in particular, in combination with other anti-malaria agents, such as chloroquine, mefloquine, pyrimethamine, sulphadoxine, or artemesinin.

The pharmaceutical compositions of the invention can be administered to a patient in need thereof. The term “patient” as used herein refers to an animal, especially a mammal. In certain particular embodiments, the mammal is a human.

In accordance with the methods and pharmaceutical compositions described herein, in yet another aspect, the invention provides PfSPP-specific antibodies and/or a signal peptide peptidase inhibitor for use in therapy for treating malaria. In certain embodiments, the PfSPP-specific antibodies are applied in conjunction with a signal peptide peptidase inhibitor in for use in therapy in treating malaria. All embodiments described herein can be applied to this aspect of the invention.

In a further aspect, the invention provides the use of PfSPP-specific antibodies in the manufacture of a medicament for treating malaria. In certain embodiments, the PfSPP-specific antibodies are combined with a signal peptide peptidase inhibitor for the manufacture of a medicament for treating malaria. All embodiments described herein can be applied to this aspect of the invention.

The Examples, which follow, are illustrative of specific embodiments of the invention, and various uses thereof. They are set forth for explanatory purposes only, and are not to be taken as limiting the invention.

### EXAMPLES

### Example 1

### Parasite Culture and Solubilization of Parasite Proteins

The P. falciparum strain 3D7 (obtained from MR4) was maintained in continuous culture in a 5% suspension of fresh type O+ human erythrocytes in RPMI 1640 at 37° C. under 5% CO₂, 5% O₂, and 90% N₂ by the method of Trager and Jensen (Trager et al., 1976, Science 193:673-5). Ring-stage parasites were synchronized by using 5% sorbitol treatment and late-stage parasites were enriched to >95% by centrifugation in 63% (v/v) Percoll as described (Goel et al., 2003, Proc Natl Acad Sci USA 100:5164-5169). The parasite protein extract was prepared by solubilizing an enriched fraction of mature parasites with an extraction buffer (50 mM Tris-HCl, pH 8.0, 150 mM NaCl, 5 mM EDTA, 5 mM EGTA, 0.5% Triton X-100, 0.5% BSA) supplemented with 2 μg/ml Aprotinin, 1 μg/ml of Leupeptin, Pepstatin A, Bestatin, 10 mM PMSF, and a cocktail of protease inhibitors (Roche, Indianapolis, Ind.). The mixture was kept on ice for 1 h and centrifuged at 12,000 g for 20 min at 4° C. The supernatant was stored at −80° C. in aliquots and used in the antibody characterization and binding assays.

### Example 2

### Identification of PfSPP as a Plasmodium Protein that Interacts with Erythrocyte Band 3

A yeast two-hybrid system was employed to identify Plasmodium proteins that interacted with human red blood cell (RBC) band 3 protein. A P. falciparum (3D7) cDNA library was screened in the yeast two-hybrid system using a peptide (5ABC) patterned on human RBC band 3 as bait (Li et al., 2004, J Biol Chem 279:5765-5771). The 5ABC amino acid sequence corresponding to residues 720-761 of human band 3 is GMPWLSATTV RSVTHANALTVMGKASTPGAAAQIQEVKEQRI. (SEQ ID NO:7). It was previously identified that band 3 interacted with P. falciparum merozoite surface protein 9 (MSP9), which existed as a co-ligand complex of MSP9-MSP1 (Kariuki et al., 2005, Biochem Biophys Res Commun 338:1690-5). It was further discovered that another P. falciparum gene product interacted strongly with the 5ABC peptide in the yeast two-hybrid assay under the highest stringency conditions. The sequence of the cDNA insert in the yeast vector was determined to be a P. falciparum signal peptide peptidase gene (1,218 bps) designated as PF14_{—}0543 in the Plasmodium Genome Database (PlasmoDB). According to PlasmoDB, the gene presumably encodes a hypothetical protein of 46.9 kDa (NP_{—}702432). Transcriptome analyses of several P. falciparum strains compiled in the PlasmoDB indicated that the gene was transcribed at a relatively high level at the late stage (Trophozoite/Schizont) of parasite intraerythrocytic life cycle. The proteome data compiled in the PlasmoDB suggest that PfSPP was expressed in merozoites.

To investigate the sequence conservation of PfSPP in various parasite strains, cDNAs were amplified from 6 strains of P. falciparum (3D7, 7G8, Dd2, HB3, K1, and FCR3, obtained from the Malaria Research and Reference Reagent Resource Center “MR4”), using primers corresponding to PfSPP sequence (Gene ID: PF14_{—}0543) and having the sequences as follows: 5′-GCCGGATCCATGAATTTATTAAAATTAATT-3′ (SEQ ID NO:8) and 5′-GCCGTCGACTCATTTATTGGTAATTCTTT-3′ (SEQ ID NO:9). No size variation was observed in the RT-PCR fragments of 6 parasite strains. PCR products were either sequenced directly or cloned in the pMAL-p2X vector (New England Biolabs, Ipswich, Mass.) for subsequent sequencing and expression.

In contrast to the information obtainable from PlasmoDB, the complete cDNA sequence of PfSPP as described herein comprises 1,239 bp encoding 412 amino acids with a predicted molecular mass of 47.6 kDa. The protein has an isoelectric point of 8.83 and the total AT content of the coding region is 72.5%. The deduced amino acid sequence of PfSPP is highly conserved among 6 strains with only a single amino acid change from alanine to serine at position 180 in the FCR3 strain (FIG. 1A). Importantly, an additional 6 amino acid residues (VFTTIL) between glycine-129 and glutamic acid-130 of the sequence set forth in the PlasmoDB were discovered in all 6 parasite strains analyzed according this the methods set forth hereon (a comparison is shown in FIG. 1A). The correct sequence with the additional 6 amino acids was not reported in the published PfSPP amino acid sequence in the PlasmoDB (3D7 strain, PF14_{—}0543). As set forth in PlasmoDB, the PfSPP gene has been analyzed to be composed of 9 exons and 8 introns. As assessed in light of the discovery of the additional amino acid sequence comprising the deduced amino acid sequence of PfSPP as described herein and not disclosed in the prior art, the first 18 nucleotides of the 4^{th} intron in PlasmoDB were incorrectly predicted and not properly included as part of the coding sequence of the 4^{th} exon of PfSPP gene.

P. falciparum PfSPP orthologues exist in different Plasmodia species including without limitation P. vivax, P. knowlesi, P. yoelii (infect rodents), P. berghei (infect non-human mammals), and P. chabaudi (infect rodents), other apicomplexa species such as Cryptosporidium parvum and Toxoplasma gondii, and many other single and multicellular eukaryotes (OrthoMCL DB, http://orthomcl.cbil.upenn.edu). However, no PfSPP paralogue was found in P. falciparum, indicating the existence of a single gene encoding PfSPP in the parasite genome. BLAST search analysis showed that PfSPP shares homology with the Presenilin-type intramembrane aspartyl proteases functionally identified in higher eukaryotes, including humans.

Primary structure analysis of PfSPP revealed conservation of aspartate-containing catalytic site motifs YD (amino acids 227-228) and LGLGD (265-269) that are required for presenilin-type intramembrane aspartyl protease activities (FIG. 1C) (Xia et al., 2003, J Cell Sci 116:2839-44). The C-terminal PALL (341-343) motif essential for a proper conformation of the active site is also present in PfSPP (Wang et al., 2006, J Neurochem 96:218-27). An uncleaved signal-anchor rather than a signal peptide sequence is predicted for PfSPP by the ConPred II program. In a previous study, PfSPP cDNA was chemically synthesized based on the incorrect cDNA structure published in the PlasmoDB, and the protein so produced showed some protease activities in mammalian cells (Nyborg et al, 2006, FASEB J 20:1671-9).

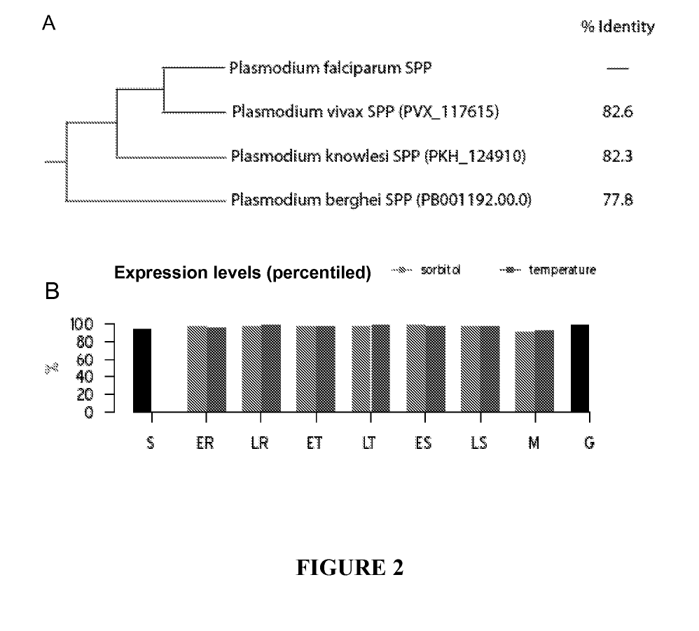

To examine whether the correct PfSPP sequence is conserved across malaria parasite species, PfSPP sequence from the 3D7 strain was compared with its counterparts in P. vivax strain SaI-1 (PVX_{—}117615) (SEQ ID NO:10), P. knowlesi strain H (PKH_{—}124910) (SEQ ID NO:11), and P. berghei strain ANKA (PB001192.00.0) (SEQ ID NO:12) (FIG. 1B). Sequence alignment of the PfSPP proteins revealed 82.6% homology with human P. vivax, 82.3% homology with monkey P. knowlesi, and 77.8% homology with mouse P. berghei (FIG. 2B). One additional Ser-384 residue was found in the P. falciparum SPP sequence whereas Asn-11 and Gln-71 residues were found only in the P. vivax and P. knowlesi SPPs but not in the P. falciparum and P. berghei SPPs (FIG. 1B). The additional 6-amino acid sequence was found between Ser-129 and Glu-130 (numbering according to the PfSPP sequence from PlasmoDB) in the SPPs of P. falciparum, P. vivax and P. knowlesi but not in the sequence from P. berghei.

The program ConPred II was used to determine the topology of PfSPP in the Plasmodium parasite membrane. The prediction accuracy of ConPred II is relatively high (˜99%), thus improving the transmembrane (TM) topological accuracy by ˜11% over other methods (Arai et al., 2004, Nucleic Acids Res 32:W390-3). Using ConPred II, the complete PfSPP amino acid was predicted to have ten transmembrane (TM) domains with preference for a cytosolic orientation of both the N- and the C-termini (FIG. 1C). Two intramembrane active site motifs (YD and LGLGD) were found located in the center of TM7 and TM8, respectively, an orientation that was consistent with the active site motifs of human SPP. The conserved PALL motif near the C-terminus of PfSPP was predicted to locate at the boundary of the TM10 region. Thus, the PfSPP topological model suggested that the malaria enzyme had characteristics similar to signal peptide peptidase but not those of presenilins. No signal peptide was predicted in the PfSPP, but an uncleaved signal-anchor sequence (Gly19-Ser38) was predicted by ConPred II.) The newly identified 6 amino acid insert of PfSPP was predicted to be located at the membrane-cytosol interface of TM4 (FIG. 1C). Because of the high sequence identity between SPPs of various malaria species (FIG. 2B), it is likely that all parasite SPPs conform to the same topology model as predicted for PfSPP.

Based on the transcriptome data obtained from PlasmoDB, the absolute transcription expression level of the intra-erythrocytic PfSPP was relatively high at the trophozoite and schizont stage of development. In comparison, the percentile expression of PfSPP as compared to all other Plasmodium gene expression was high (>90%) at all intraerythrocytic stages including the rings, trophozoites, and schizonts (FIG. 2B). The PfSPP gene was also expressed in the gametocytes and sporozoites (FIG. 2B).

### Example 3

### Production of PfSPP-Specific Antibodies and Characterization of the PfSPP Protein

The cDNA insert in the yeast two-hybrid screen encoded the C-terminus of PfSPP (amino acids 183-412) containing two or three putative extracytosolic regions (FIG. 1C). One extracellular region, termed ER, contained 18-26 amino acid residues depending on the topology model, whereas the other two regions contained only a few residues. To establish the existence of the PfSPP corresponding to full-length sequence, anti-peptide polyclonal antibodies were raised in rabbits against a segment in the PfSPP/ER.

Anti-peptide antibodies against the PfSPP/ER region were produced. A short peptide corresponding to residues 246-264 (EAPVKLLFPVSSDPVHYSM, SEQ ID NO:4) of P. falciparum (3D7) PfSPP was synthesized with an additional cysteine residue at the N-terminus and conjugated to Keyhole Limpet Hemocyanin, KLH, through a disulfide bond. Peptide-specific antibodies raised in rabbits were harvested by affinity purification of serum on a cyanogen bromide-immobilized peptide. The affinity-purified antibodies, hereinafter referred to as the PfSPP/ER antibody, stored at 4° C. in PBS, were tested for specific reactivity against recombinant PfSPP/ER and native P. falciparum PfSPP by immunoblotting.

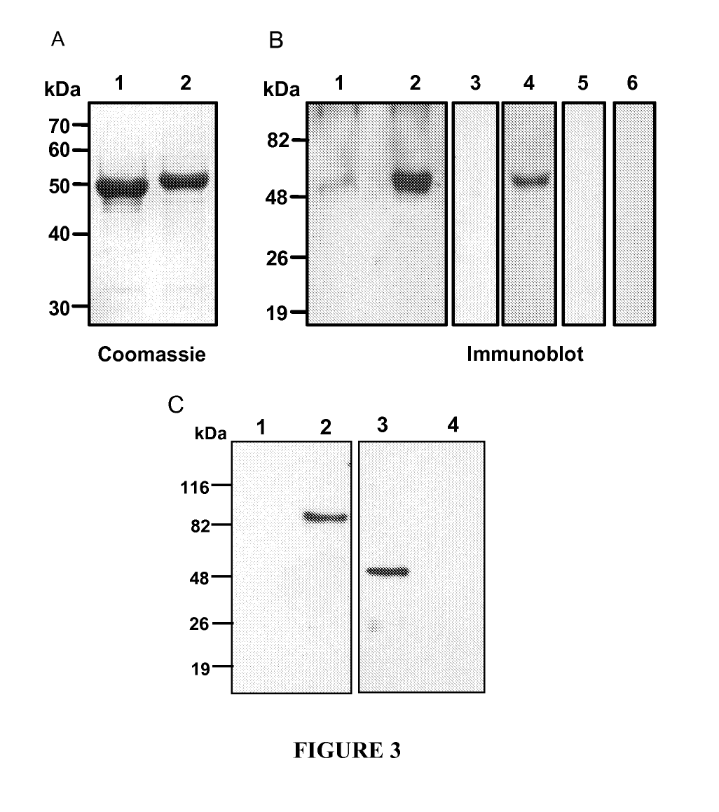

The ER domain of PfSPP was produced as a fusion to the maltose binding protein (MBP-PfSPP/ER) by PCR cloning (FIG. 3A) to be used to verify the specificity of the antibodies. The PfSPP gene fragment encoding amino acids 226-266 was amplified by PCR from P. falciparum (3D7) genomic DNA and cloned into pMAL-c2x (NE Biolabs). The PCR primers used were 5′-CGC GAATTCGTATATGATATTTTCTGG-3′ (sense, EcoRI, SEQ ID NO:13) and 5′-CGCTCTAGAACCAAGCATAC TGTAATG-3′ (antisense, XbaI, SEQ ID NO:14). The recombinant PfSPP/ER domain was expressed in Escherichia coli DH5α as a fusion to the maltose binding protein (MBP). MBP-PfSPP/ER was affinity-purified on amylose resin. Mono-specific polyclonal PfSPP/ER antibodies (pAbs) that were affinity-purified using an immunogen-peptide column reacted specifically to recombinant PfSPP/ER domain in the immunoblot assay (FIG. 3B, lane 2). These antibodies reacted to a specific protein migrating at ˜47 kDa as a single band in the immunoblot of P. falciparum (3D7) protein extract containing a mixture of native parasite proteins (FIG. 3B, lane 4). These results suggest that P. falciparum PfSPP was expressed during the blood stage of parasite development.

Anti-peptide polyclonal antibodies against the PfSPP C-terminal region were also produced. A short peptide corresponding to residues 393-412 (EIPKIQETPVSNAKKRITNK, SEQ ID NO:5) of P. falciparum (3D7) PfSPP was synthesized with an additional cysteine residue at the N-terminus and conjugated to Keyhole Limpet Hemocyanin, KLH, through a disulfide bond. Peptide-specific antibodies were raised in rabbits and the polyclonal antiserum was purified by affinity purification on a cyanogen bromide-immobilized antigen peptide. The affinity-purified antibodies, hereinafter referred to as the PfSPP C-terminal antibodies, stored at −20° C. in PBS with 50% glycerol, were tested for specific reactivity against recombinant PfSPP and native P. falciparum PfSPP by immunoblotting.

As shown in FIG. 3C, mono-specific anti-PfSPP C-terminus pAb reacted specifically to the recombinant MBP-PfSPP (lane 2) and native P. falciparum PfSPP (lane 3), but not with MBP (lane 1) and human ghosts (lane 4). Lane 1, MBP; lane 2, MBP-PfSPP; lane 3, P. falciparum extract; lane 4, human RBC ghosts.

### Example 4

### Localization of PfSPP in P. falciparum

Protein co-localization studies using the anti-PfSPP/ER pAbs were carried out to determine the expression characteristics of PfSPP in mature parasites. Synchronized P. falciparum (3D7) schizonts were smeared and air dried on glass slides and fixed with 100% methanol for 30 min at −20° C. Slides were incubated for 2 h with a mixture of two antibodies containing affinity purified mono-specific anti-PfSPP rabbit Abs (1:1000) and anti-EBA-175 rat pAb (1:1000; MR4), directed against a Plasmodium175 kDa erythrocyte binding antigen, anti-MSP1 mouse mAb 5.2 (1:1000; MR4), directed against merozoite surface protein 1, or anti-RAP1 mouse mAb (1:1000; MR4), directed against Plasmodium rhoptry-associated protein 1. After three washings in PBS-T (0.1% Tween-20 in PBS), samples were incubated for 1 h with appropriate secondary Abs (Molecular Probes/Invitrogen, Carlsbad, Calif.) conjugated with either Alexa Fluor 488 (green fluorescence) or Alexa Fluor 594 (red fluorescence). Dual-color fluorescence images were captured using a microscope (Zeiss LSM510, Germany) equipped with a digital camera at 100× magnification.

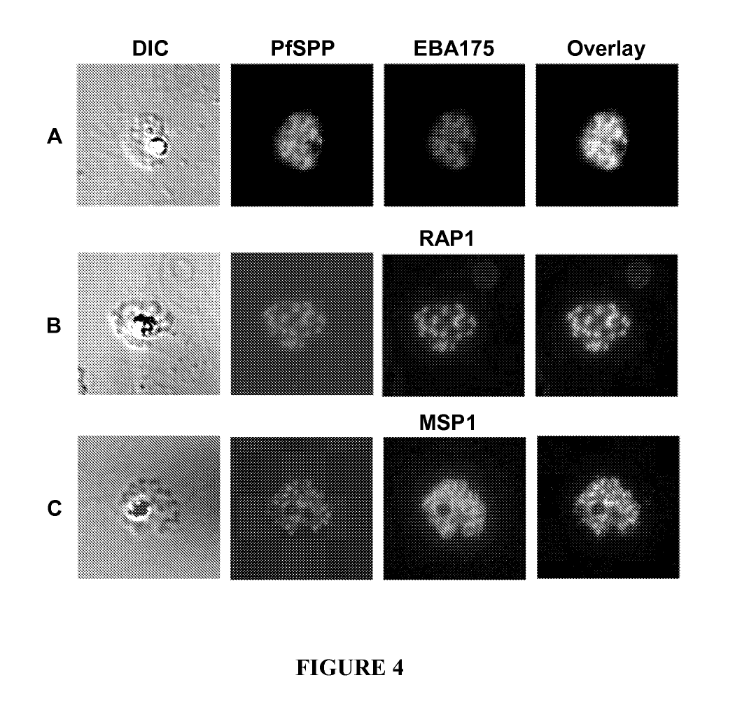

An indirect immunofluorescence assay (IFA) using P. falciparum schizonts showed that PfSPP co-localized with EBA-175 (FIG. 4). There appeared to be some overlap of immunofluorescence with RAP1 presumably due to the close proximity of the two organelles, but there was no overlap with MSP1 (FIG. 4). These results suggested that PfSPP was expressed in the micronemes of mature parasites.

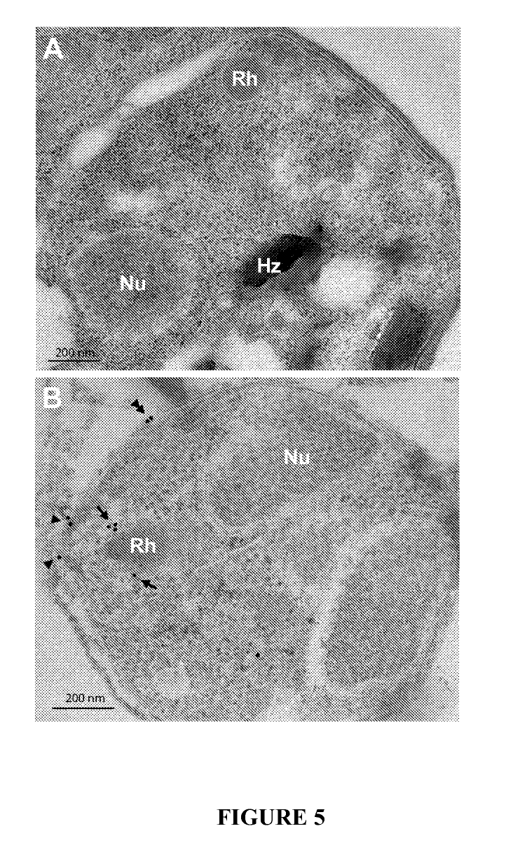

PfSPP has been recently detected in the merozoites as described by Florens et al., 2002, Nature 419:520-6. Immunogold electron microscopy was performed to determine the localization of PfSPP in the internal structure of late stage schizonts. Late-stage schizonts (40-48 h post invasion) enriched from a synchronized P. falciparum culture were washed in RPMI 1640, fixed with 4% paraformaldehyde and 0.1% glutaraldehyde for 1 h at 4° C. in 0.1 M sodium phosphate buffer (pH 7.2), and embedded in White London Resin. Ultra-thin sections were blocked in PBS containing 1% BSA and incubated with affinity purified anti-PfSPP/ER pAb diluted in the above-described solution. Samples were washed and incubated with secondary antibodies conjugated with gold particles (10 or 15 nm diameter) at a 1:10 dilution for 1 h. Labeled sections were stained with uranyl acetate and lead citrate, and observed using a Philips FEI Tecnai F30 transmission electron microscope at 300 kV. While control preimmune sera did not show any labeling of merozoites (FIG. 5A), the anti-PfSPP/ER antibody showed specific labeling of the micronemes and the apical surface of merozoites (FIG. 5B). Consistent with the IFA results, the immunogold particles were not detected in the parasite rhoptries. These results suggest that PfSPP appears to be expressed in micronemes and on the apical surface of the merozoites. Evidence from mass spectrometry has also confirmed the expression of PfSPP in the merozoites. See Florens et al., 2002, Nature 419:520-6. Together, these observations suggested a dynamic nature of PfSPP polypeptide trafficking between various cellular compartments in infected erythrocytes.

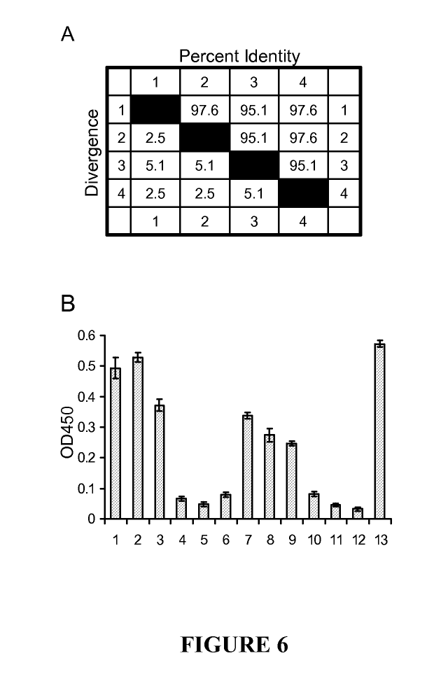

The putative surface exposed PfSPP/ER region is highly conserved, with the P. falciparum sequence having 97.6% sequence identity with cognate SPP proteins from P. vivax and P. knowlesi, and 95.1% sequence identity with SPP protein from P. berghei (FIG. 6A). To investigate whether the sequences were conserved or variable in PfSPP/ER from malaria patients, parasite genomic DNA was isolated from 64 blood samples of field isolates, and the DNA encoding the PfSPP/ER region within exons 8 and 9 were amplified, using the forward primer ACAGTCTGGTTTGTTTGTATATGA (SEQ ID NO:15) and reverse primer CTGGTATAATAATATCTCCTAAACCAAGC (SEQ ID NO:16). PCR products were sequenced with sequencing primers ATACATATTAATTGTTCTTGTT (SEQ ID NO:17) and TTGAAGCTCCAGTAAAATTG (SEQ ID NO:18), and analysed for polymorphisms using the BioEdit alignment program (North Carolina State University).

Direct sequencing of PCR products revealed only a single synonymous mutation in the codon of serine-256 changing the codon from TCG to TCC in seven field isolates. These results indicated that the PfSPP/ER exofacial loop sequence was highly conserved, and non-synonymous mutations were not tolerated within this part of the gene. To determine if antibodies against the PfSPP/ER region existed in malaria patients, recombinant MBP-PfSPP/ER fusion protein was used to detect serum antibodies against PfSPP in 10 malaria patients living in rural village of Kambila, Mali where transmission of P. falciparum is seasonal and intense (mean age 38 years, range 28-51 years). An ELISA screen revealed high plasma reactivity in 6 malaria patients and low plasma reactivity in 3 patients, indicating>70% plasma-positivity against PfSPP. The response was specific, as little serum reactivity was observed in individuals who had never been exposed to malaria (FIG. 6B). These results suggest that the PfSPP/ER region was highly conserved, and was exposed to the immune system at some stage of malaria infection.

### Example 5

### Anti-PfSPP Antibodies Blocked Erythrocyte Invasion

The function of PfSPP in RBC invasion was evaluated using mono-specific polyclonal anti-PfSPP/ER Abs to block P. falciparum invasion of RBCs in culture. A fraction enriched with late-stage P. falciparum trophozoites was washed twice with RPMI 1640 and returned to the culture by incubating the parasites with fresh RBCs in a 96-well microplate (final: 3% hematocrit, 2% parasitemia). An aliquot of affinity purified anti-PfSPP Abs and pre-immune IgG were added to the culture medium as an inhibitor to give 0, 12.5, 25, 50, and 100 μg/ml final concentration in a volume of 200 μl. The sample with no antibodies, i.e., PBS only, served as a negative control. Rabbit pre-immune serum was used at 10% dilution as another negative control. Parasite samples were incubated for 24 h under standard culture conditions and assays were carried out in triplicate. Each sample was analyzed by counting ring-stage parasites in Giemsa-stained thin smears. Approximately 2,000 RBCs were counted for each sample to quantify parasitemia, from which the percent inhibition of invasion was calculated relative to the control sample with no Abs, based on the formula: (parasitemia for no Abs control−parasitemia for sample containing Abs)/parasitemia for no Abs control×100%. Data are presented as mean±s.d.

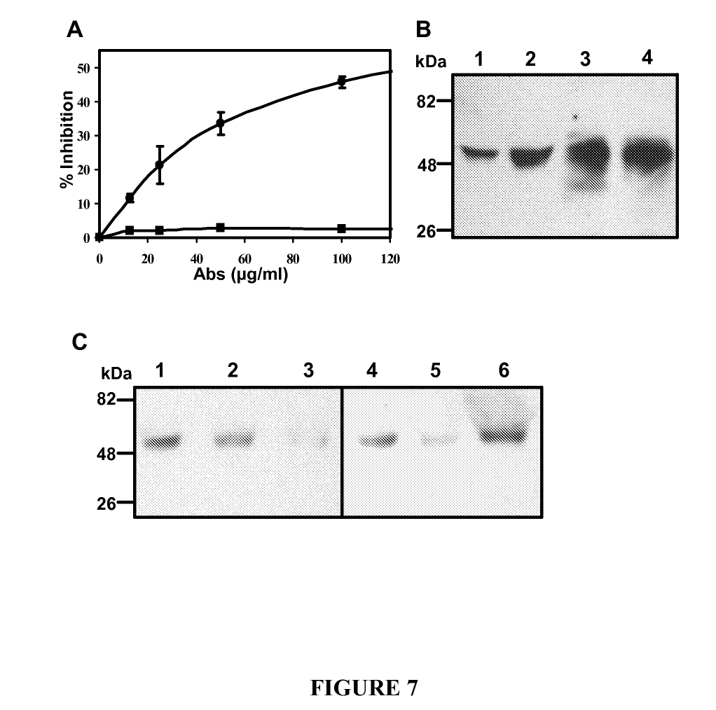

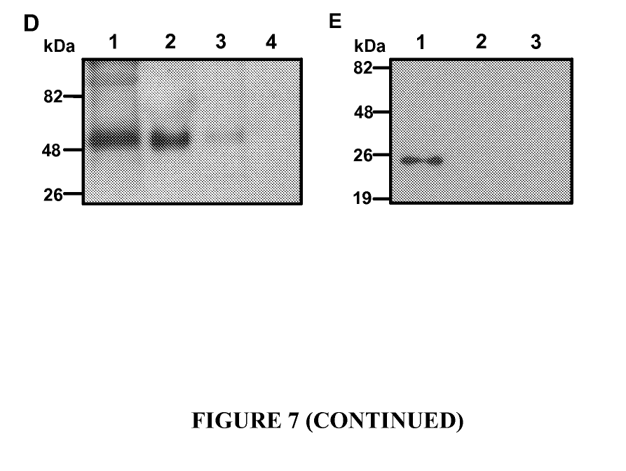

This invasion inhibition assay showed that anti-PfSPP/ER Abs added to the culture medium blocked P. falciparum from invading RBCs (FIG. 7A). The invasion blocking effect was concentration-dependent, showing over 45% invasion inhibition at 100 μg/ml antibody concentration as compared to the PBS (no antibodies) control. Anti-PfSPP antibodies did not affect parasites maturation since trophozoites and schizonts did not accumulate in the assay samples (data not shown). The inhibition rate was determined relative to the no-antibody control taken as 100% invasion (0% inhibition). Preimmune sera added at 10% dilution showed 2.0±1.1% invasion inhibition, which is considered relatively insignificant compared to the anti-PfSPP/ER Abs samples. Total IgG isolated from preimmune sera and added up to 100 μg/ml also showed insignificant inhibitory effect on parasite invasion (2.6±0.4%). Therefore, it is unlikely that inhibition of RBC invasion by anti-PfSPP/ER Abs was simply a consequence of steric interference because the inhibitory effect was seen only with PfSPP-specific antibodies. Together, these results suggest that PfSPP present on the surface of the intact merozoites interacted with the anti-PfSPP/ER Abs in the culture medium, the interaction of which can interfere PfSPP binding to band 3 of RBC and affect RBC invasion.

### Example 6

### PfSPP Binding to Red Blood Cells

To carry out a RBC-binding assay in solution using native PfSPP, a spent culture supernatant of P. falciparum containing native PfSPP was prepared as follows. Trophozoites enriched from a 5% sorbitol-synchronized P. falciparum culture were allowed to mature for 16 h to form schizonts and merozoites. The parasites were left in the culture flask to lyse and the proteins were released into the culture supernatant. Aliquots of the culture were centrifuged at either 12,000 g for 20 min or 40,000 g for 15 min at 4° C. The culture supernatant and pellet were analyzed by immunoblot analysis using anti-PfSPP pAbs. The culture supernatant prepared at 12,000 g was stored at −80° C. in aliquots and used in subsequent RBC binding assays. In these assays, a 500 μl sample of P. falciparum culture supernatant was added to untreated, trypsin-treated, chymotrypsin-treated, or neuraminidase-treated intact human RBCs (50 μl packed volume) for 1 h at room temperature. The cells were centrifuged at 12,000 g for 30 s through a layer of silicone oil (500 μl). Proteins bound to RBCs were eluted by incubating the RBCs in 1.5 M NaCl (20 μl) for 30 min at room temperature. Salt-eluted proteins were analyzed by SDS-PAGE followed by immunoblotting using anti-PfSPP Abs. The pretreatment of intact RBCs with enzymes was carried out as described (Goel et al., 2003, Proc Natl Acad Sci USA 100:5164-5169). To examine the inhibitory effect of 5ABC on protein binding, soluble GST-5ABC (40 μM) was added to the normal RBC sample. The GST (40 μM) protein served as a negative control.

As shown in the immunoblot as depicted in FIG. 7B, full-length PfSPP was found in the supernatant fraction obtained by ultracentrifugation at 40,000 g (lane 1) and high-speed centrifugation at 12,000 g (lane 2). Presumably, PfSPP present in the supernatant fractions was mostly present in small vesicular form as a significant reduction of PfSPP upon ultracentrifugation was observed (lane 1). Consistent with this observation, a higher amount of full-length PfSPP was associated with the pellet fractions as compared with the supernatant in both centrifuged samples (FIG. 7B; lanes 3 and 4). These biochemical properties are consistent with what would have been expected for a transmembrane protein (FIG. 1C).

A solution-binding assay using the P. falciparum culture supernatant prepared by high-speed centrifugation (12,000 g) was carried out to further investigate potential interactions between PfSPP and human RBCs. Immunoblotting of the RBC sample using anti-PfSPP/ER Abs showed that native PfSPP was associated with intact RBCs (FIG. 7C, lane 1). To investigate whether PfSPP interacted with the 5ABC domain of band 3, a fusion protein of GST-5ABC peptide corresponds to residues 720-761 of human band 3 (SEQ ID NO:7) was prepared as described (Li et al., 2004, J Biol Chem 279:5765-5771).

When soluble GST-5ABC was added to the binding assay, the binding of PfSPP to RBCs was decreased by 94% as quantified by densitometry (lane 3). To determine the enzyme-sensitivity of the receptor responsible for PfSPP binding to intact RBCs, human RBCs pretreated with trypsin, chymotrypsin, and neuraminidase were used in the binding assays as previously described (Goel et al., 2003, Proc Natl Acad Sci USA 100:5164-5169). Immunoblotting showed that the association of native PfSPP to RBCs was reduced in the chymotrypsin-treated RBCs by 84% (FIG. 7C, lane 5), while it remained relatively unchanged in the trypsin-treated (lane 4) and neuraminidase-treated (lane 6) RBCs as compared to the normal RBC sample (lane 1). It was known that the extracellular regions of human band 3 in intact RBCs are sensitive to chymotrypsin, but remain resistant to the other two enzymes (Goel et al., 2003). Together, the results suggested that the human band 3 receptor was involved in the association of native PfSPP to human RBCs in suspension.

### Example 7

### Direct Binding of PfSPP to the 5ABC Domain of Band 3

Since native PfSPP is insoluble, the RBC binding assay could not distinguish whether PfSPP bound directly to intact RBCs or through other proteins that are likely to be present in the vesicles containing PfSPP. Moreover, a direct involvement of host band 3 in mediating direct association of PfSPP to RBCs was also not conclusive. To address this issue, bead-binding assays using native PfSPP solubilized in the non-ionic detergent Triton X-100 (P. falciparum protein extract) and using GST-5ABC beads were carried out. GST-5ABC and GST conjugated to GSH beads (30-60 μl) were incubated with solubilized P. falciparum proteins (200 μl) in binding buffer (PBS, 1 mM EDTA, 0.1% w/v Triton X-100, and 0.5 mg/ml BSA) at 4° C. overnight (600 μl final volume). The resulting beads were sedimented by centrifugation, washed three times with binding buffer without BSA, and analyzed by SDS-PAGE followed by immunoblotting using affinity-purified anti-PfSPP/ER pAbs.

Immunoblot analysis showed that PfSPP solubilized in 1% Triton X-100 bound specifically to the 5ABC domain of band 3 immobilized on beads (FIG. 7D). In addition, direct interaction of PfSPP with a soluble fragment of band 3 using purified recombinant proteins, MBP-PfSPP/ER (conjugated to beads) and Trx-5ABC (soluble protein) was tested. The MBP-PfSPP/ER fusion conjugated to amylose beads was incubated at 4° C. overnight (350 μl final volume) with soluble Trx-5ABC (pET Trx expression system, Novagen, Inc./EMD, Gibbstown, N.J.) in binding buffer (50 mM Tris-HCl, pH 7.0, 150 mM NaCl, 1 mM EDTA, 0.1% Triton X-100, and 0.5 mg/ml BSA). Beads were sedimented by low speed centrifugation and washed three times with PBS containing 0.1% Tween 20 (PBS-T). Trx-5ABC bound to the beads was detected by immunoblotting using anti-His-HRP mAb (Santa Cruz Biotechnology, Santa Cruz, Calif.). MBP on Ni-NTA beads and soluble Trx were used as negative controls.

The 5ABC domain of band 3 interacted specifically with the PfSPP/ER domain in the immunoblot assay (FIG. 7E). These results demonstrated that the putative ER segment of parasite PfSPP bound directly to the 5ABC region of RBC band 3. Together, these data suggested that native PfSPP interacted with human RBCs during P. falciparum invasion.

### Example 8

### PfSPP's Role in Malaria Parasite Invasion

To investigate the role of PfSPP in parasite invasion, a chemical approach was adopted by employing three synthetic inhibitors of mammalian membrane aspartyl proteases. The inhibitor (Z-LL)₂-ketone (Calbiochem/EMD Biosciences, Gibbstown, N.J.) is specific for mammalian SPP, whereas the L-685,458 and DAPT inhibitors (both were gift of Dr. S. Sisodia of the University of Chicago) are known to be specific for the γ-secretase/Presenilin-1 complex (FIG. 8A). (Z-LL)₂-ketone does not inhibit the γ-secretase/Presenilin-1 activity in live mammalian cells up to a concentration of 100 μM (Weihofen et al., 2003, J Biol Chem 278:16528-33). In contrast, L-685,458 inhibited both SPP and γ-secretase/Presenilin-1, whereas DAPT had no effect on SPP activity at 100 μM (Weihofen et al., 2003). A prior study demonstrated that in vitro enzyme activity of an incomplete version of PfSPP lacking the 6 amino acids was inhibited by the SPP inhibitor (Z-LL)₂-ketone and LY411,575 (Nyborg 1,2006, FASEB J20:1671-9). Herein, human erythrocytes with highly synchronized P. falciparum schizonts were incubated in the presence of increasing concentrations of (Z-LL)₂-ketone, L-685,458, and DAPT. After 20 h of incubation, the rings were counted from the inhibitor and DMSO treated samples. The DAPT inhibitor had no effect on parasite invasion, whereas (Z-LL)₂-ketone and L-685,458 caused a significant decrease in the number of new rings in a dose-dependent manner (FIG. 8B). Parasite cultures incubated with 10 μM of (Z-LL)₂-ketone and L-685,458 resulted in >95% inhibition of new ring formation with no accumulation of the schizonts (FIG. 8C). These results suggested that the enzymatic activity of PfSPP was essential for efficient merozoite invasion of human erythrocytes.

### Example 9

### PfSPP's Role in Malaria Parasite Growth

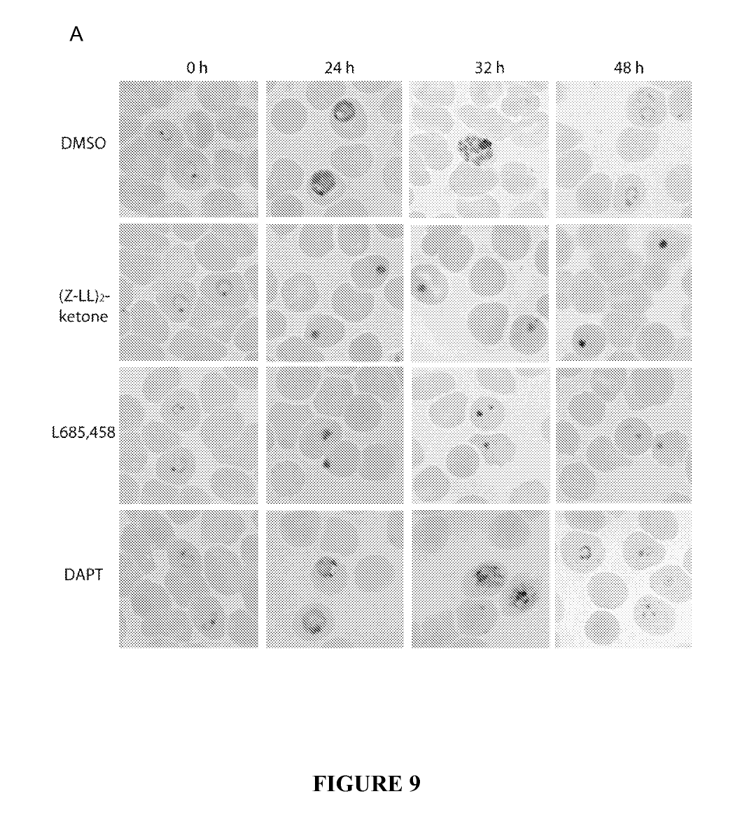

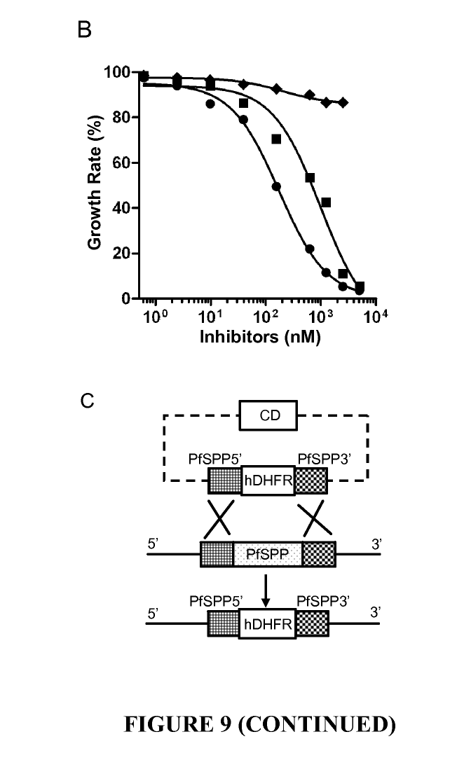

The effects of the three inhibitors on malaria parasite growth in human erythrocytes were examined Synchronized ring stage parasites were incubated with inhibitors in the presence of 0.2% DMSO. Both (Z-LL)₂-ketone and L-685,458 caused a significant inhibition of parasite growth in erythrocytes, whereas DAPT had no effect (FIG. 9A). A complete inhibition of parasite replication was observed at 10 μM of (Z-LL)₂-ketone and L-685,458 with no detection of ring stage parasites after one cycle of asexual multiplication (FIG. 9B). The precipitous decline in parasitemia presumably originated from inhibition of late rings or early trophozoites, as pyknotic parasites were observed after 24-48 h of culture in the presence of (Z-LL)2-ketone and L-685,458 (FIG. 4). Destruction of the inhibitor-treated parasites was irreversible, as no progression to the second asexual cycle was observed. In contrast, DAPT had little or no effect on parasite growth as compared to the DMSO-treated control (FIG. 9A). Both (Z-LL)₂-ketone and L-685,458 inhibited 3D7 parasite growth in a dose-dependent manner with IC50 values of 0.985 μM and 0.174 μM, respectively (FIG. 9B).

Since the primary structure of PfSPP is highly conserved, the effect of these inhibitors on the growth of other P. falciparum strains was also tested. The parasite strains tested included two chloroquine-resistant P. falciparum strains (7G8, Dd2), one chloroquine-sensitive strain (HB3), and one mildly chloroquine-resistant strain (FCR3). Parasite growth was evaluated in these four strains by measuring [³H]-hypoxanthine incorporation starting at 24 h post-invasion. Both (Z-LL)₂-ketone and L-685,458 inhibited parasite growth in all 4 parasite strains with the IC50 values similar to 3D7 strain, while DAPT had no effect on any strain (Table 2).

TABLE 2
In vitro inhibition of P. falciparum growth by PfSPP
inhibitors as measured by radioactive incorporation assay

| | IC50 ± SE (μM) | | |
|---|---|---|---|
| Parasite strain | (Z-LL)2 ketone | L685,458 | DAPT |
| Dd2 | 1.08 ± 0.07 | 0.18 ± 0.03 | >10 |
| 7G8 | 1.12 ± 0.05 | 0.21 ± 0.04 | >10 |
| FCR3 | 1.23 ± 0.04 | 0.23 ± 0.03 | >10 |
| HB3 | 1.06 ± 0.05 | 0.19 ± 0.02 | >10 |

Each assay was repeated at least two times. IC₅₀ values are means ± standard errors (SE) of the mean for experiments run in triplicate

### Example 10

### Unsuccessful Attempts to Disrupt the PfSPP Gene in P. falciparum Indicating that PfSPP Gene is Essential to the Parasite

To examine if the single PfSPP gene in the Plasmodium genome is essential for parasite development, attempts were made to genetically knockout the PfSPP gene in P. falciparum (FIG. 9C). To disrupt the PfSPP gene in 3D7 strain, 5′ and 3′ segments of the PfSPP gene were cloned into the P. falciparum transfection plasmid pCC-1 to generate the pCC-1ΔPfSPP vector (FIG. 9C). The 5′ segment (616 bp) was PCR amplified from genomic DNA (3D7) using primers 5′-GGCTTCCGCGGATGAATTTATTAAAATTAAT-3′ (SEQ ID NO:19) and 5′TACAGCTTAAGAGTAAGCAAAGCTGCAGATC-3′ (SEQ ID NO:20), and was cloned into the SacII and AflII sites of pCC-1 upstream of the hDHFR cassette (a gift from Dr. Cowman, see Maier et al., 2008, Cell 134(1):48-61). The 3′ segment (711 bp) of PfSPP was amplified using the primers 5′-GCCGAATTCTCTGGTTTGTTTGTATATG-3′ (SEQ ID NO:21) and 5′-GCCGAATTCTCATTTATTGGTAATTCTTT-3′ (SEQ ID NO:22), and cloned downstream of the hDHFR cassette. The underlined sequences indicate restriction sites used for cloning. PfSPP gene could be disrupted via double crossover mediated homologous recombination between the chromosomal PfSPP locus and the knockout plasmid pCC-1ΔPfSPP. A cytosine deaminase (CD) cassette was used for negative selection. Ring-stage parasites were transfected with 100 μg of pCC-1ΔPfSPP plasmid in a 0.2-cm cuvette using Gene Pulser (Bio-Rad, Hercules, Calif.) at 0.31 kV, 950 μF, using maximum resistance. WR99210 (5 nM, DHFR inhibitor) was added 48 h after the electroporation, and maintained thereafter. No live parasites were observed for up to 35 days in the presence of WR99210. Multiple attempts to disrupt the PfSPP gene were unsuccessful, suggesting an essential role of this protease in the erythrocytic life cycle of malaria parasite.

It should be understood that the foregoing disclosure emphasizes certain specific embodiments of the invention and that all modifications or alternatives equivalent thereto are within the spirit and scope of the invention as set forth in the appended claims.

What is claimed is:

1. An isolated nucleic acid comprising a polynucleotide sequence
(a) that is identified by SEQ ID NO:1;
(b) that encodes a polypeptide comprising the amino acid sequence as identified by SEQ ID NO:2, or
(c) that is complementary to the nucleotide sequence of (a) or (b).

2. The isolated nucleic acid of claim 1, comprising a polynucleotide sequence that encodes a polypeptide having the amino acid sequence as identified by SEQ ID NO:2.

3. A membrane preparation comprising a polypeptide having the amino acid sequence as identified by SEQ ID NO:2 having Plasmodium falciparum signal peptide peptidase (PfSPP) activity.

4. An expression vector comprising the polynucleotide of claim 2.

5. A host cell comprising the expression vector of claim 4.

6. The host cell of claim 5 wherein the host cell is a bacteria cell, a mammalian cell, a yeast cell or an insect cell.

7. A method of expressing a Plasmodium falciparum signal peptide peptidase (PfSPP) polypeptide comprising the step of culturing the host cell of claim 6 under suitable conditions to express the polypeptide.

8. The method of claim 7, wherein the polypeptide comprises an amino acid sequence identified by SEQ ID NO:2.

9. A purified antibody or an antigen-binding fragment thereof that specifically binds to a Plasmodium falciparum signal peptide peptidase (PfSPP) comprising the amino acid sequence identified by SEQ ID NO:2, wherein the antibody or antigen-binding fragment thereof recognizes an epitope located within amino acid residues 246-264 of SEQ ID NO:2 or within amino acid residues 393-412 of SEQ ID NO: 2.

10. The antibody of claim 9 wherein the purified antibody or antigen-binding fragment thereof recognizes an epitope located within amino acid residues 246-264 of SEQ ID NO: 2.

11. The antibody of claim 9 wherein the purified antibody or antigen-binding fragment thereof recognizes an epitope located within amino acid residues 393-412 of SEQ ID NO: 2.

12. The antibody of any one of claims 9-11 wherein the antibody is a monoclonal antibody or antigen-binding fragment thereof.

13. The antibody of claim 12 wherein the antibody is a humanized, human, chimeric, or CDR-grafted antibody, or an antigen-binding fragment thereof.

14. The antibody of claim 9 or 10 or antigen-binding fragment thereof that inhibits the binding of a Plasmodium falciparum signal peptide peptidase to an erythrocyte.

15. The antibody of claim 14, wherein the antibody or antigen-binding fragment thereof inhibits the binding of a Plasmodium falciparum signal peptide peptidase to the erythrocyte surface protein band 3.

16. A method of inhibiting a Plasmodium parasite invasion of a cell, comprising contacting the Plasmodium parasite with the antibody of claim 9 or 10.

17. The method of claim 16, wherein the Plasmodium parasite is Plasmodium falciparum.

18. The method of claim 17, wherein the cell is an erythrocyte.

19. The method of claim 18, wherein the antibody or antigen-binding fragment thereof recognizes an epitope located within amino acid residues 246-264 of SEQ ID NO: 2.

20. A method of inhibiting a Plasmodium parasite replication, growth or development in a cell comprising contacting the cell with the antibody of claim 9 or 10.

21. The method of claim 20, wherein the Plasmodium parasite is Plasmodium falciparum.

22. The method of claim 20 wherein the antibody or antigen-binding fragment thereof recognizes an epitope located within amino acid residues 246-264 of SEQ ID NO: 2.

23. The method of claim 20 further comprising contacting the cell with an effective amount of a signal peptide peptidase inhibitor.

24. The method of claim 23, wherein the signal peptide peptidase inhibitor is (Z-LL)₂-ketone, LY411575, NVP-AHW700-NX or L685,458.

25. A method of treating or preventing malaria in a human in need thereof comprising administering to the human an effective amount of the purified antibody of claim 9 or antigen-binding fragment thereof.

26. The method of claim 25 wherein the antibody or antigen-binding fragment thereof recognizes an epitope located within amino acid residues 246-264 of SEQ ID NO: 2.

27. The method of claim 25, further comprising administering to the human an effective amount of a signal peptide peptidase inhibitor.

28. The method of claim 27, wherein the signal peptide peptidase inhibitor is (Z-LL)₂-ketone, LY411575, NVP-AHW700-NX or L685,458.

29. A method of inhibiting a Plasmodium parasite invasion of a cell comprising contacting the Plasmodium parasite with a signal peptide peptidase inhibitor.

30. The method of claim 29, wherein the signal peptide peptidase inhibitor is (Z-LL)₂-ketone, LY411575, NVP-AHW700-NX or L685,458.

31. The method of claim 29, further comprising contacting the cell with the antibody of claim 9 or 10 or antigen-binding fragment thereof.

32. The method of claim 29, wherein the Plasmodium parasite is Plasmodium falciparum.

33. A method of inhibiting a Plasmodium parasite replication, growth or development in a cell comprising contacting the cell with a signal peptide peptidase inhibitor.

34. The method of claim 33, wherein the signal peptide peptidase inhibitor is (Z-LL)₂-ketone, LY411575, NVP-AHW700-NX or L685,458.

35. The method of claim 33, further comprising contacting the cell with the antibody of claim 9 or antigen-binding fragment thereof.

36. The method of claim 33, wherein the Plasmodium parasite is Plasmodium falciparum.

37. A method of treating or preventing malaria in a human in need thereof comprising administering to the human an effective amount of a signal peptide peptidase inhibitor.

38. The method of claim 37, further comprising administering to the human an effective amount of the antibody of claim 9 or antigen-binding fragment thereof.

39. The method of claim 38, wherein the signal peptide peptidase inhibitor is (Z-LL)₂-ketone, LY411575, NVP-AHW700-NX or L685,458.

40. A pharmaceutical composition for inhibiting or preventing malaria comprising the purified antibody of claim 9 or antigen-binding fragment thereof and at least one pharmaceutically acceptable carrier, diluent and excipient.

41. The pharmaceutical composition of claim 40 further comprising a signal peptide peptidase inhibitor.

42. The pharmaceutical composition of claim 41 wherein the signal peptide peptidase inhibitor is (Z-LL)₂-ketone, LY411575, NVP-AHW700-NX or L685,458.

43. A pharmaceutical composition for inhibiting or preventing malaria comprising a signal peptide peptidase inhibitor and the purified antibody of claim 9 or antigen-binding fragment thereof and at least one pharmaceutically acceptable carrier, diluent and excipient.

44. The pharmaceutical composition of claim 43 wherein the signal peptide peptidase inhibitor is (Z-LL)₂-ketone, LY411575, NVP-AHW700-NX or L685,458.

45. A kit for treating or preventing malaria comprising the pharmaceutical composition of any one of claims 40-44 and optionally instructions for use.

46. A method of screening for a compound that inhibits Plasmodium falciparum signal peptide peptidase (PfSPP) activity comprising the steps of contacting the polypeptide of claim 3 with a test compound and a substrate that is converted by the PfSPP activity, wherein a decrease in the levels of substrate conversion as compared to control indicates that the compound is an inhibitor of the PfSPP activity.

47. A method of detecting or quantifying PfSPP protein in a sample comprising the steps of:
(a) contacting the sample with the antibody of claim 9; and
(b) detecting the binding of the PfSPP protein in the sample to the antibody.

48. A method of detecting a Plasmodium parasite in a sample by detecting a Plasmodium signal peptide peptidase (SPP) protein in the sample comprising the steps of:
(a) contacting the sample with the antibody of claim 9; and
(b) detecting the binding of the Plasmodium SPP protein in the sample to the antibody,
wherein the binding of the Plasmodium SPP protein to the antibody indicates that the Plasmodium parasite is in the sample.

49. The method of claim 48, wherein the Plasmodium parasite is Plasmodium falciparum.

50. A method of diagnosing Plasmodium infection in a human comprising the steps of:
(a) contacting a sample obtained from the human with the antibody of claim 9; and
(b) assaying the sample for a Plasmodium SPP polypeptide binding of the antibody,
wherein binding of the Plasmodium SPP polypeptide to the antibody indicates Plasmodium infection in the human.

51. The method of claim 50, wherein the Plasmodium is Plasmodium falciparum.

52. A kit for detecting the presence of a Plasmodium parasite in a sample comprising the antibody of claim 9 and optionally instructions for use.

53. A kit for diagnosing Plasmodium infection in a human comprising the antibody of claim 9 and optionally instructions for use.

54. A malaria vaccine comprising the polypeptide of claim 3 or an antigenic fragment thereof and a pharmaceutical carrier, diluent or excipient.

55. The vaccine of claim 54, wherein the antigenic fragment comprises amino acid residues 246-264 of the sequence as identified by SEQ ID NO:2.

56. A composition comprising the polypeptide of claim 3 or an antigenic fragment thereof and a pharmaceutical carrier, diluent or excipient.

57. The composition of claim 56, wherein the antigenic fragment comprises amino acid residues 246-264 of the sequence as identified by SEQ ID NO:2.

58. The composition of claim 57 that is a malaria vaccine.

59. A method of immunizing a human in need thereof against Plasmodium infection or malaria comprising the step of administering the malaria vaccine of any one of claims 54, 55 and 58 to the human.

60. The method of claim 59 wherein the Plasmodium is Plasmodium falciparum.
